(19) United States
(12) Patent Application Publication
(10) Pub. No.: US 2021/0001141 A1
(43) Pub. Date: Jan. 7, 2021

(54) PHOTOBIOMODULATION THERAPY SYSTEMS AND DEVICES

(71) Applicant: Joovv, Inc., San Clemente, CA (US)

(72) Inventors: Greg Pfiffner, San Clemente, CA (US); Jakob Kishon, San Clemente, CA (US)

(21) Appl. No.: 17/027,472

(22) Filed: Sep. 21, 2020

Related U.S. Application Data
(63) Continuation-in-part of application No. 17/027,338, filed on Sep. 21, 2020.
Application No. 17/027,338 is a continuation-in-part of application No. 16/904,243, filed on Jun. 17, 2020.
Application No. 16/904,243 is a continuation-in-part of application No. 16/598,033, filed on Oct. 10, 2019.
Application No. 16/598,033 is a continuation of application No. 16/584,784, filed on Sep. 26, 2019, now Pat. No. 10,639,495.
Application No. 16/584,784 is a continuation of application No. 16/227,289, filed on Dec. 20, 2018, now Pat. No. 10,478,635.
Application No. 16/227,289 is a continuation-in-part of application No. 16/167,385, filed on Oct. 22, 2018.
(60) Provisional application No. 62/872,835, filed on Jul. 11, 2019.
(60) Provisional application No. 62/863,247, filed on Jun. 18, 2019.

(51) Int. Cl. A61N 5/06

(52) U.S. Cl. CPC A61N 5/06; A61N 2005/0659; A61N 2005/0663; A61N 2005/0629; A61N 2005/064

(57) ABSTRACT

A light therapy device may comprise a housing comprising a front bezel and a back portion detachably coupled to the front bezel, a printed circuit board assembly coupled to the housing, and at least one LED electrically coupled to the printed circuit board assembly. The at least one LED may be arranged and configured to emit light through at least one aperture of the front bezel. In some embodiments, the at least one LED is arranged and configured to pulse at a predetermined frequency and a predetermined duty cycle.

### CROSS-REFERENCE TO RELATED APPLICATIONS

The entire contents of the following application are incorporated by reference herein: U.S. Provisional Patent Application No. 62/460,113; filed Feb. 17, 2017; and entitled THERAPEUTIC LIGHT SOURCE AND HANGING APPARATUS.

The entire contents of the following application are incorporated by reference herein: U.S. Non-Provisional patent application Ser. No. 15/616,028; filed Jun. 7, 2017; and entitled THERAPEUTIC LIGHT SOURCE AND HANGING APPARATUS.

The entire contents of the following application are incorporated by reference herein: PCT Patent Application No. PCT/US2018/018288; filed Mar. 1, 2018; and entitled THERAPEUTIC LIGHT SOURCE AND MOUNTING APPARATUS.

The entire contents of the following application are incorporated by reference herein: U.S. Non-Provisional patent application Ser. No. 16/167,385; filed Oct. 22, 2018; and entitled PHOTOBIOMODULATION THERAPY SYSTEMS AND METHODS.

The entire contents of the following application are incorporated by reference herein: PCT Patent Application No. PCT/US2019/057292; filed Oct. 21, 2019; and entitled PHOTOBIOMODULATION THERAPY SYSTEMS AND METHODS.

The entire contents of the following application are incorporated by reference herein: U.S. Non-Provisional patent application Ser. No. 16/227,289; filed Dec. 20, 2018; and entitled PHOTOBIOMODULATION THERAPY SYSTEMS AND METHODS.

The entire contents of the following application are incorporated by reference herein: U.S. Provisional Patent Application No. 62/863,247; filed Jun. 18, 2019; and entitled PHOTOBIOMODULATION THERAPY SYSTEMS AND METHODS.

The entire contents of the following application are incorporated by reference herein: U.S. Provisional Patent Application No. 62/872,835; filed Jul. 11, 2019; and entitled PHOTOBIOMODULATION THERAPY SYSTEMS AND METHODS.

The entire contents of the following application are incorporated by reference herein: U.S. Non-Provisional patent application Ser. No. 16/584,784; filed Sep. 26, 2019; and entitled PHOTOBIOMODULATION THERAPY SYSTEMS AND METHODS.

The entire contents of the following application are incorporated by reference herein: U.S. Non-Provisional patent application Ser. No. 16/598,033; filed Oct. 10, 2019; and entitled PHOTOBIOMODULATION THERAPY SYSTEMS AND METHODS.

The entire contents of the following application are incorporated by reference herein: PCT Patent Application No. PCT/US2019/059845; filed Nov. 5, 2019; and entitled PHOTOBIOMODULATION THERAPY SYSTEMS AND METHODS.

The entire contents of the following application are incorporated by reference herein: U.S. Non-Provisional patent application Ser. No. 16/904,243; filed Jun. 17, 2020; and entitled PHOTOBIOMODULATION THERAPY SYSTEMS AND METHODS.

The entire contents of the following application are incorporated by reference herein: PCT Patent Application No. PCT/US2020/038187; filed Jun. 17, 2020; and entitled PHOTOBIOMODULATION THERAPY SYSTEMS AND METHODS.

The entire contents of the following application are incorporated by reference herein: U.S. Non-Provisional patent application Ser. No. 17/027,338; filed Sep. 21, 2020; and entitled PHOTOBIOMODULATION THERAPY DEVICE ACCESSORIES.

### BACKGROUND

### Field

Various embodiments disclosed herein relate to photobiomodulation therapy devices. Certain embodiments relate to light therapy devices configured to emit at least one of red and infrared light.

### Description of Related Art

Photobiomodulation therapy (or light therapy) is a therapeutic technique that uses low-level wavelengths of light to improve health and treat a variety of health conditions, including skin issues, such as wrinkles, scars, and persistent wounds, among many other conditions. Photobiomodulation therapy uses non-ionizing light sources, including lasers, light emitting diodes, and/or broadband light, in the visible (400-700 nm) and infrared (700-1100 nm) electromagnetic spectrum. Photobiomodulation is a nonthermal process involving endogenous chromophores eliciting photophysical (i.e. linear and nonlinear) and photochemical events at various biological scales. Similar to how plants use sunlight to heal and grow, humans and animals are able to harness these wavelengths of light and turn them into cellular energy. This treatment stimulates the body's natural healing processes.

Currently, there are a number of photobiomodulation therapy devices available on the market. However, many of these devices are too small and require multiple sessions to treat large areas. As a result, there is a need for a photobiomodulation therapy system that can treat several areas in fewer treatments.

### SUMMARY

The disclosure includes a light therapy device comprising a housing comprising a front bezel and a back portion detachably coupled to the front bezel, a printed circuit board assembly coupled to the housing, and at least one LED electrically coupled to the printed circuit board assembly, wherein the at least one LED is arranged and configured to emit light through at least one aperture of the front bezel. In some embodiments, the front bezel is configured to create a gradient to pull heat from the printed circuit board assembly and thereby act as a heat sink for the light therapy device. The front bezel may be comprised of aluminum. In some embodiments, an interior surface of the front bezel is coated with a white surface finished to thereby reflect light.

The light therapy device may further comprise at least one lens coupled to the front bezel, the at least one lens positioned to cover at least a portion of the at least one aperture such that the light travels from the at least one LED through the at least one lens. The device may further comprise at least one reflector coupled to the front bezel, wherein the at least one reflector surrounds at least a portion of the at least one LED to thereby reflect the light emitted from the at least one LED. In some embodiments, each LED of the at least one LED comprises a multi-die chip LED. The multi-die chip LED comprises four LEDs. In many embodiments, the four LEDs comprise two crimson LEDs and two infrared LEDs. The at least one LED may be dimmable.

In some embodiments, the printed circuit board assembly comprises the at least one LED and a control board. The control board may include at least one integrated circuit driver. In some embodiments, the light therapy device further comprises a rechargeable battery that includes a protection circuit module. The light therapy device may be configured to simultaneously charge the rechargeable battery and emit light from the at least one LED.

In some embodiments, the light therapy device further comprises a rubber handle coupled to a side portion of the housing and configured to enable a user to hold the light therapy device. The housing may define a soap-bar shape ergonomic form factor.

The light therapy device may be arranged and configured to operate in a recovery plus mode, wherein when the light therapy device operates in the recovery plus mode, the at least one LED may be arranged and configured to emit pulsed near infrared light. In some embodiments, the light therapy device is arranged and configured to operate in an ambient mode, wherein when the light therapy device operates in the ambient mode, the at least one LED is arranged and configured to emit ambient light. The light therapy device may be arranged and configured to operate in an alarm clock mode, wherein when the light therapy device operates in the alarm clock mode, the at least one LED is arranged and configured to emit ambient light at at least one of a predetermined time and upon an occurrence of a predetermined condition. When the light therapy device operates in the alarm clock mode, the at least one LED may be arranged and configured to emit ambient light of increasing intensity.

The light therapy device may be at least substantially waterproof.

The disclosure includes a light therapy system comprising a housing, a printed circuit board assembly coupled to the housing, and at least one LED electrically coupled to the printed circuit board assembly, wherein the at least one LED is arranged and configured to emit light through at least one aperture of the housing, wherein the at least one LED is arranged and configured to pulse at a predetermined frequency and at a predetermined duty cycle.

The light therapy system may further comprise a control panel coupled to the housing and operatively coupled to at least one of the printed circuit board assembly and the at least one LED. In some embodiments, the control panel further comprises a power button arranged and configured to operatively control power to the light therapy device. The control panel may further comprise a pulse button arranged and configured to operatively control the pulse of the at least one LED. In some embodiments, the control panel further comprises at least one of a mode button, a play/pause button, and a time button, wherein the mode button is arranged and configured to select a mode for light emission from the at least one LED, the play/pause button is arranged and configured to at least one of start and stop light emission from the at least one LED, and the time button is arranged and configured to select a time period for light emission from the at least one LED. The control panel may further comprise at least one indication light, wherein the at least one indication light is arranged and configured to indicate at least one of a power status, a red light emission status, an infrared light emission status, and a pulse status.

In some embodiments, the control panel comprises a controller board arranged and configured to operate the control panel, the controller board communicatively coupled to a mobile application on a remote computing device, the mobile application arranged and configured to operate the light therapy device by sending at least one command to the controller board. The control panel may further comprise a beeper function arranged and configured to emit a sound after a predetermined amount of time.

In many embodiments, the light therapy device is a first light therapy device arranged and configured to operate in a lead mode, the system further comprising a second light therapy device arranged and configured to operate in a follow mode. The first light therapy device may be arranged and configured to enter the lead mode upon pressing the power button for at least a predetermined amount of time. In some embodiments, when the first light therapy device is in the lead mode and the second light therapy device is in the follow mode, the second light therapy device is configured to be controlled via the control panel of the first light therapy device. When the first light therapy device is in the lead mode, the control panel may be active and when the second light therapy device is in the follow mode, a control panel of the second light therapy device may be inactive.

In some embodiments, the printed circuit board assembly comprises the at least one LED and at least one integrated circuit driver. The light therapy system may further comprise a medical grade power supply unit at least one of electrically and communicatively coupled to the power button, the power supply unit configured to output about 48 volts of power. In some embodiments, the medical grade power supply unit is electrically coupled to at least one insulation displacement connector configured to receive at least one of power and data from a plurality of electrical wires coupled to the medical grade power supply unit.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments. For ease of viewing the figures, not all features may be labeled in each drawing.

FIG. 1 illustrates a front perspective view of a light therapy device, according to some embodiments.

FIG. 2 illustrates a perspective view of a front bezel of a light therapy device, according to some embodiments.

FIG. 3 illustrates a front view of a light therapy device including a printed circuit board assembly, according to some embodiments.

FIGS. 4A and 4B illustrate front and back views, respectively, of a printed circuit board assembly, according to some embodiments.

FIG. 5 illustrates a schematic view of at least one LED, according to some embodiments.

FIG. 6 illustrates a front view of a light therapy device, according to some embodiments.

FIG. 7 illustrates a side view of a light therapy device, according to some embodiments.

FIG. 8 illustrates a back view of a light therapy device, according to some embodiments.

FIG. 9 illustrates a bottom perspective view of a light therapy device, according to some embodiments.

FIG. 10 illustrates a perspective view of a charging stand, according to some embodiments.

FIG. 11 illustrates a perspective view of a rechargeable battery, according to some embodiments.

FIGS. 12 and 13 illustrate front perspective views of a light therapy system, according to some embodiments.

FIG. 14 illustrates a graph of a predetermined duty cycle, according to some embodiments.

FIG. 15 illustrates a front view of a printed circuit board assembly, according to some embodiments.

FIG. 16 illustrates a front view of a control panel, according to some embodiments.

FIG. 17 illustrates a front view of a controller board, according to some embodiments.

FIG. 18 illustrates a schematic view of a control panel 116 and a remote computing device, according to some embodiments.

FIG. 19 illustrates a back view of a light therapy device, according to some embodiments.

FIG. 20 illustrates a back view of a plurality of light therapy devices, according to some embodiments.

FIGS. 21A and 21B illustrate front views of a control panel in a lead mode and a follow mode, respectively, according to some embodiments.

FIG. 22 illustrates a perspective view of a light therapy system, according to some embodiments.

FIG. 23 illustrates a schematic view of various electrical components of a light therapy device, according to some embodiments.

FIGS. 24, 25, 26, 27, and 28 illustrate electrical schematics of a light therapy system, according to some embodiments.

FIG. 29A illustrates a front view of a light therapy device, according to some embodiments.

FIG. 29B illustrates a front view of at least one insulation displacement connector, according to some embodiments.

FIG. 29C illustrates a top view of a plurality of electrical wires that pass through the at least one insulation displacement connector, according to some embodiments.

FIG. 29D illustrates a top and side view of an AC input socket, according to some embodiments.

FIG. 30 illustrates a perspective view of various electrical components of a light therapy system, according to some embodiments.

FIG. 31 illustrates a perspective view of at least one insulation displacement connector, according to some embodiments.

FIG. 32 illustrates a perspective view of a various components of a light therapy device, according to some embodiments.

FIG. 33 illustrates a perspective view of a power supply unit, according to some embodiments.

FIG. 34 illustrates a perspective view of a back plate of a power supply unit, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. Additionally, the structures, systems, and/or devices described herein may be embodied as integrated components or as separate components.

For purposes of comparing various embodiments, certain aspects and advantages of these embodiments are described. All such aspects or advantages are not necessarily achieved by any particular embodiment. For example, various embodiments may be carried out in a manner that achieves or optimizes one advantage or group of advantages as taught herein without necessarily achieving other aspects or advantages as may also be taught or suggested herein.

### INTRODUCTION

An objective of the present invention is to provide a variety of photobiomodulation therapy devices. Of the embodiments described herein are devices of different sizes and comprising different numbers of red and/or infrared LEDs.

### LIST OF REFERENCE NUMERALS

10—light therapy device
12—housing
14—front bezel
16—back portion
17—at least one coupling peg
18—printed circuit board assembly
20—at least one LED
22—at least one aperture
24—heat sink
26—interior surface
28—at least one lens
30—at least one reflector
32—multi-die chip LED
34—control board
38—rechargeable battery
39—plurality of conductor cables
40—protection circuit module
41—connector
42—handle
44—side portion
46—soap-bar shape
48—charging stand
49—bottom portion
50a—charging contact (on light therapy device)
50b—charging contact (on charging stand)
100—light therapy system
102—light therapy device
103—stand
104—housing
105—treatment surface
106—printed circuit board assembly
108—at least one LED
110—at least one aperture
112—predetermined frequency
114—predetermined duty cycle
116—control panel
118—power button
120—pulse button
122—mode button
124—play/pause button
126—time button
128—at least one indication light
130—control panel display
132—controller board
134—mobile application
136—remote computing device
138—at least one command
140—lead mode
142—follow mode
144—at least one cable
146—at least one IC driver
148—power supply unit
149—back plate
150—at least one connector
151—AC input socket
152—plurality of electrical wires
153a—male connector
153b—female connector
154—back portion
156—plurality of ports
158—heat sink
160—flexible printed circuit connector
162—fan connector

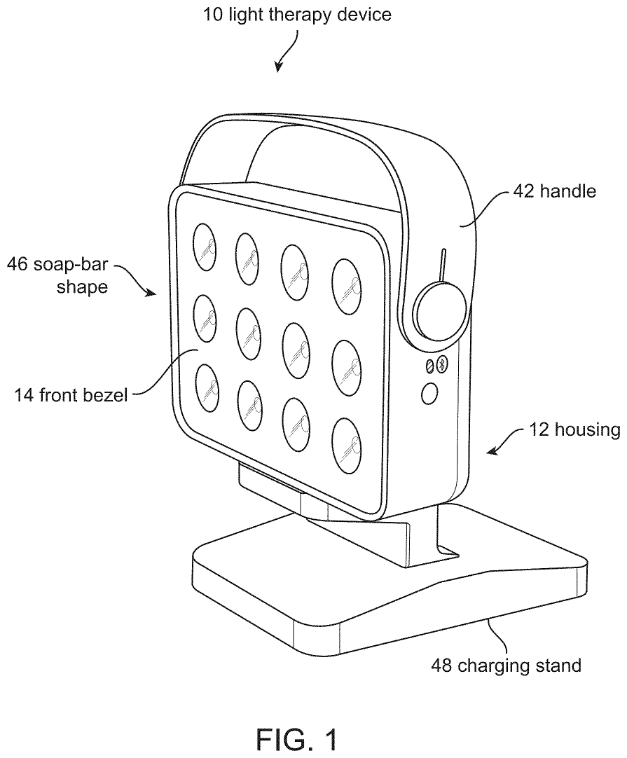

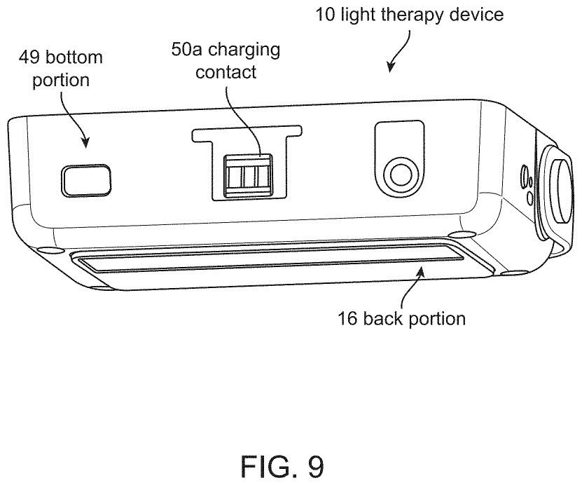

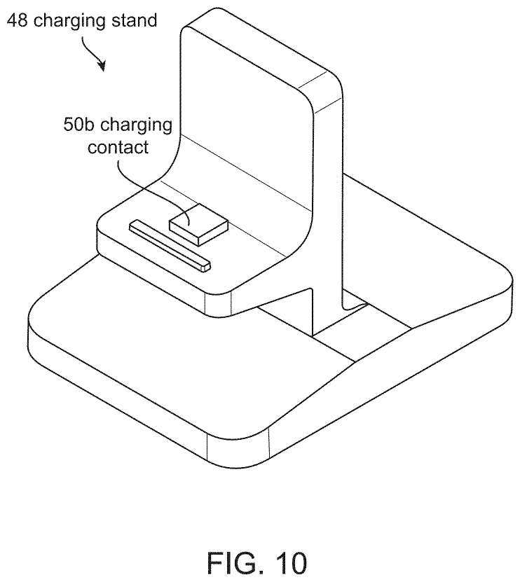

FIG. 1 illustrates a perspective view of a light therapy device 10. As shown, the device 10 may include a housing 12, a front bezel 14, and a handle 42. The device 10 may be configured to couple to a charging stand 48, as demonstrated in FIG. 1. Further details of the charging stand 48 will be discussed later in the disclosure, particularly with reference to FIGS. 9 and 10. FIG. 1 also shows that the device 10 may comprise a soap-bar shape 46. In some embodiments, the soap-bar shape 46 comprises a substantially rectangular shape with rounded corners. The device 10 may comprise any ergonomic shape suitable for a user to carry the device 10. Though not shown in FIG. 1, in some embodiments, the device 10 includes a grip, a second handle, or a similar holding mechanism coupled to the housing 12. The device 10 may define dimensions of about 3.75 inches by about 5.75 inches, and may weigh about 1 pound.

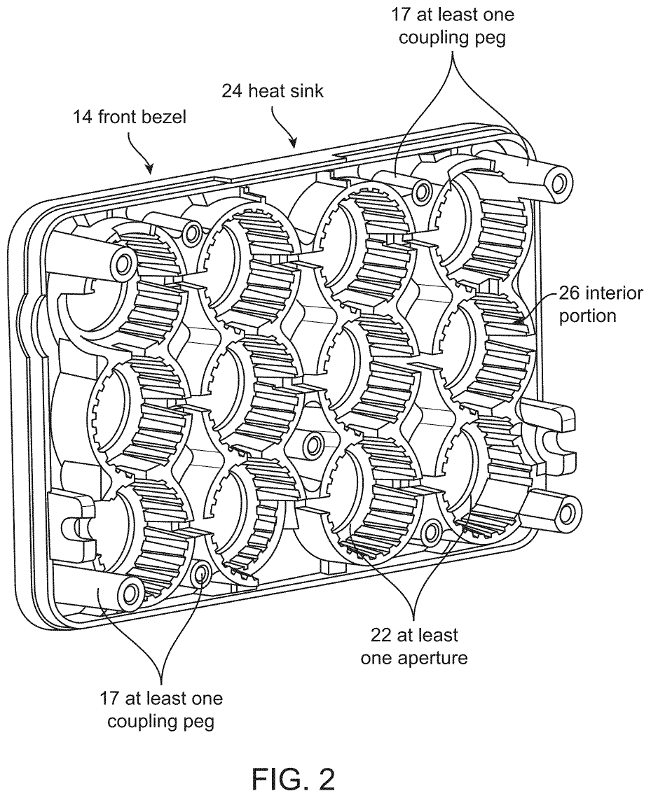

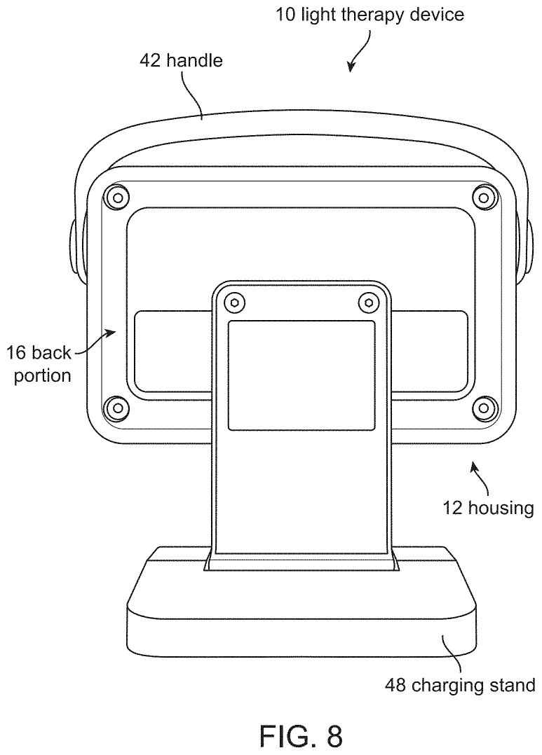

FIG. 2 shows a back perspective view of the front bezel 14 detached from the light therapy device. In many embodiments, the housing 12 comprises the front bezel 14 and a back portion 16, which is shown in FIG. 8. The front bezel 14 may be configured to detachably couple to the back portion 16 via the at least one coupling peg 17, shown in FIG. 2. In some embodiments, the front bezel 14 is configured to fixedly couple to the back portion 16 via the at least one coupling peg 17. The back portion 16 may comprise mechanisms configured to receive the at least one coupling peg 17. In some embodiments, as shown in FIG. 2, the at least one coupling peg 17 is substantially hollow. As such, the back portion 16 may comprise at least one protrusion or similar mechanism, wherein the at least one coupling peg 17 is configured to receive the at least one protrusion or similar mechanism within the hollow portion of the peg 17. In some embodiments, a mechanical fastener, such as a screw, passes through each of the at least one coupling peg 17 to thereby threadably mechanically couple to the back portion 16. It should be appreciated that any type of mechanical fastener may be used.

The front bezel 14 may comprise any number of pegs, such as two pegs, three pegs, four pegs, five pegs, six pegs, seven pegs, eight peds, nine pegs, ten pegs, eleven pegs, twelve pegs, and thirteen or more pegs. Each peg of the at least one coupling peg 17 may define the same length. As shown in FIG. 2, the at least one coupling peg 17 may comprise varying lengths. In some embodiments, the front bezel 14 is configured to couple to the back portion 16 via other coupling methods, in addition to or instead of the at least one coupling peg 17. For example, the front bezel 14 may couple to the back portion 16 via a channel lock, a friction fit, a draw latch or other latching mechanism, and/or any other suitable mechanical coupling mechanism.

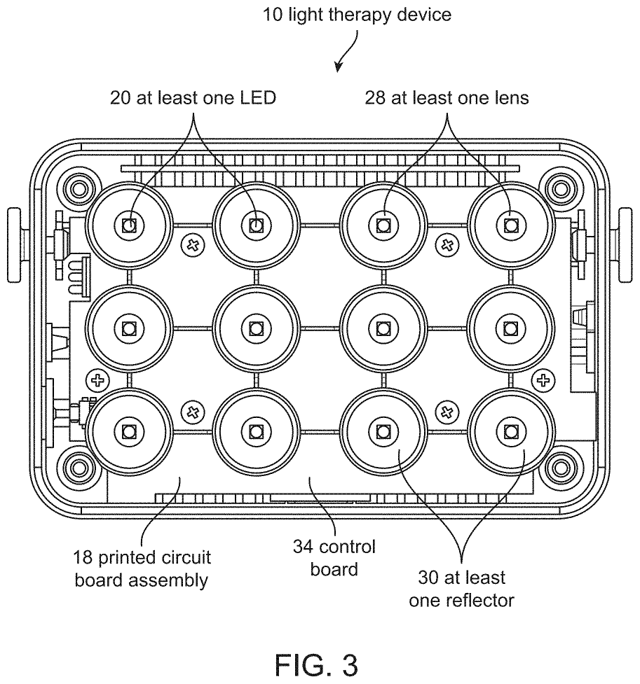

The front bezel 14 may include at least one aperture 22. As will be discussed further with reference to FIG. 3, the at least one aperture 22 may comprise at least one of at least one lens 28 and at least one reflector 30 configured to fit over at least one LED 20. In many embodiments, the front bezel 14 is configured to act as a heat sink 24 for the device 10. As such, the front bezel 14 may create a gradient to pull heat from the printed circuit board assembly 18 (shown in FIG. 3 and hereafter referred to as the “PCBA 18”) and disperse the heat to an external environment around the device 10. The front bezel 14 may be constructed of at least one of aluminum, magnesium, aluminum alloy, and magnesium alloy. The use of these, or similar, materials may facilitate the transfer of heat from the PCBA 18 through the front bezel 14. Generally, the front bezel 14 may be constructed of any type of material commonly used in the art of heat sinks. In some embodiments, the front bezel 14 comprises substantially the entire front portion of the device 10. Accordingly, substantially the entire front portion of the device 10 may act as the heat sink 24. The total heat dissipation of the device 10 may be about 6.6 W.

Due to the use of the front bezel 14 as a heat sink 24, in many embodiments, the device 10 does not require the use of one or multiple fans or ventilation openings for the release of heat. As such, the device 10 may be considered substantially waterproof. In some embodiments, the device 10 meets the requirements for an Ingress Protection Code rating of 22, also known as an IP22 rating. An IP22 rating may indicate that the device 10 is “Protected from touch by fingers and objects greater than 12 millimeters” and “Protected from water spray less than 15 degrees from vertical,” as defined by the International Electrotechnical Commission.

Referring now to FIG. 3, as previously mentioned, the at least one aperture 22 comprises at least one lens 28 and at least one reflector 30. In many embodiments, the at least one lens 28 is configured to cover at least a portion of the at least one aperture 22 such that light travels from at least one LED 20 of the device 10 through the at least one lens 28. The at least one reflector 30 may be configured to surround at least a portion of the at least one LED 20 to thereby reflect the light emitted from the at least one LED 20. In some embodiments, the front bezel 14 comprises both the at least one lens 28 and the at least one reflector 30. As such, the front bezel 14 may be considered a “dual-purpose” element of the system 10. (And when the function as a heat sink 24 is included, the front bezel 14 may be considered a “tri-purpose” element.) In some embodiments, an interior portion 26 (shown in FIG. 2) of the front bezel 14 is coated with a white surface finish to thereby reflect light.

The at least one reflector 30 may comprise the portion of the front bezel 14 surrounding each LED of the at least one LED 20 and each lens of the at least one lens 28. For example, going back to FIG. 2, each reflector of the at least one reflector 30 may comprise one of the circular cavities of the front bezel 14. In some embodiments, each cavity is positioned around one LED of the at least one LED 20. Each cavity may be at least partially covered, for example on a front surface of the front bezel 14, with a lens of the at least one lens 28. An interior portion 26 of each reflector of the front bezel 14 may be coated with a white surface finish to thereby reflect light. In some embodiments, the interior portion 26 comprises another reflective surface coating, such as chrome. The at least one reflector 30 may be configured to concentrate the light emitted from the at least one LED 20 and direct the concentrated light toward the at least one lens 28.

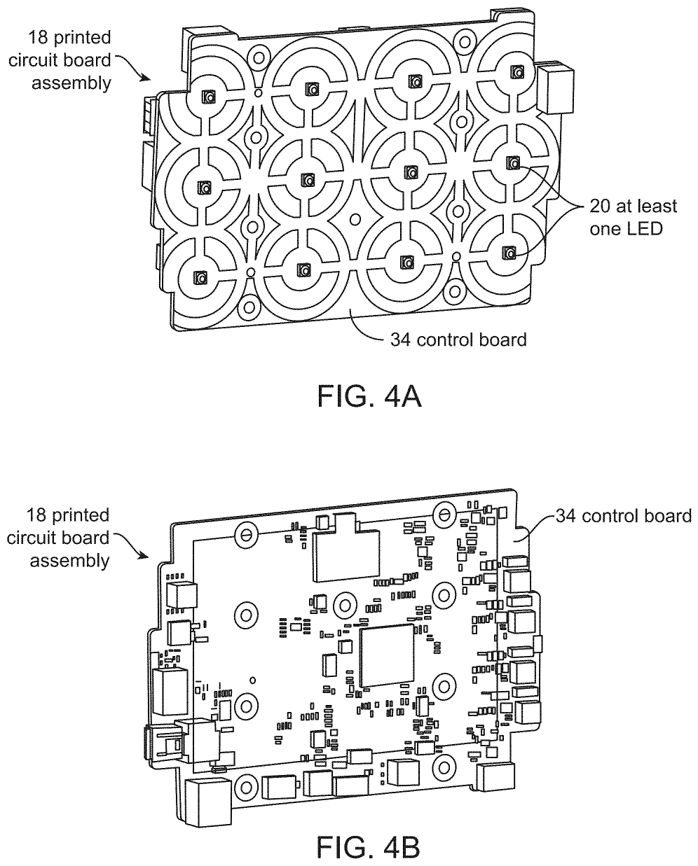

FIGS. 4A and 4B show front and back views, respectively, of the PCBA 18. In many embodiments, the PCBA 18 is coupled to the housing 12 and located in an interior portion of the housing 12 between the front bezel 14 and the back portion 16. The at least one LED 20 may be electrically coupled to the PCBA 18. In some embodiments, the PCBA 18 also comprises a control board 34 operatively coupled to the at least one LED 20 and at least one user control on the housing 12. As such, the PCBA 18 may comprise a single board design that integrates the at least one LED 20 and the control board 34 into the single board. The PCBA 18 may also include the LED drivers, switches, and MCU logic. In some embodiments, the PCBA 18 is coupled to a Bluetooth module configured to facilitate Bluetooth connection between the device 10 and a remote computing device. The remote computing device may include at least one user control operatively coupled to the control board 34 and, thereby, to the at least one LED 20. In some embodiments, the PCBA 18 comprises a single multilayer FR4 board. Though not shown in FIGS. 4A and 4B, the PCBA 18 may include at least one integrated circuit driver.

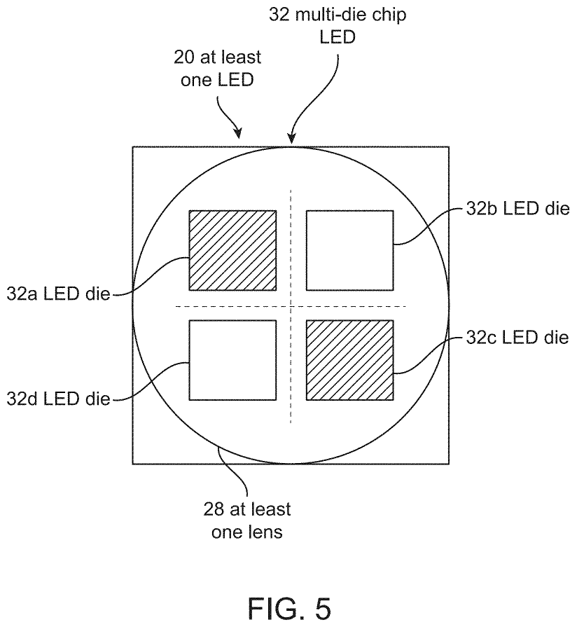

FIG. 5 shows a schematic view of at least one LED 20. In many embodiments, each LED of the at least one LED 20 comprises a multi-die chip LED 32. As demonstrated in FIG. 5, each multi-die chip LED 32 may comprise four LED die 32a, 32b, 32c, and 32d. In some embodiments, two of the four LED die, for example LED die 32a and 32c, comprise LEDs configured to emit red light at a wavelength of about 660 nm. The other two LED die, 32b and 32d, may comprise LEDs configured to emit infrared light at a wavelength of about 850 nm. In some embodiments, the LED die configured to emit red light are configured to emit red light at a wavelength between 655 nm and 660 nm with an irradiance of about 8.3 mW/cm² at 6 inches. The LED die configured to emit infrared light may be configured to emit infrared light at a wavelength between about 840 nm and 870 nm with an irradiance of about 13.75 mW/cm² at 6 inches. It should be noted that irradiance measurements are approximate and based on measurements taken at a room temperature of 25° C. The total unit radiant flux may be equal to or greater than 5,099 mW. The total light output may be about 8.09 W. In some embodiments, the multi-die chip LED 32 comprises part number PBLB-3LQE-MJ produced by ProLight Opto, of Epistar Corp. (Taiwan). Wavelengths for light emission, for example, 660 nm and 850 nm, may be selected by the user independently or in combination with each other on a session-by-session basis.

The device 10 may include a total of twelve LEDs in the at least one LED 20. Each LED of the twelve may comprise one multi-die chip LED 32. As such, the total number of LED die coupled to the PCBA 18 may comprise forty-eight LED die. In some embodiments, the at least one LED 20 comprises at least one 4-in-1 LED in a 3030 package. Stated differently, each LED of the at least one LED 20 defines a width of 3.0 mm and a length of 3.0 mm. Each LED of the at least one LED 20 may define a height of about 1.8 mm. The red and infrared LEDs may define a cross pattern as shown in FIG. 5, where the shaded LED die 32a and 32c may be considered red LEDs, and the non-shaded LED die 32b and 32d may be considered infrared LEDs. In some embodiments, LEDs configured to emit the light at the same wavelength are located side-by-side or top-to-bottom. The following tables illustrate additional technical details of the at least one multi-die chip LED 32.

| | | |
|---|---|---|
| LED Type | IR (850 nm) | Red (660 nm) |

| | | | | |
|---|---|---|---|---|
| LED Vf | 1.5 | V | 2.0 | V |
| LED Current/Loop | 175 | mA | 175 | mA |
| LED Power | 263 | mW | 350 | mW |

| | | |
|---|---|---|
| Total PCBA | IR (850 nm) | Red (660 nm) |

| | | |
|---|---|---|
| No. LED/Loop | 12 | 12 |

| | | | | |
|---|---|---|---|---|
| Loop Voltage | 18.0 | V | 24.0 | V |
| Loop Current | 175 | mA | 175 | mA |

| | | |
|---|---|---|
| No. of Parallels | 2 | 2 |

| | | | | |
|---|---|---|---|---|
| Driver Current | 350.0 | mA | 350.0 | mA |

| | | |
|---|---|---|
| Driver Efficiency | 93% | 7% |

| | | | | |
|---|---|---|---|---|
| Total LED Power | 6.30 | W | 8.40 | W |

In some embodiments, the at least one LED is configured to emit light at wavelengths other than 660 nm and 850 nm. For example, at least one die in the multi-die chip LED 32 may be configured to emit light at any of the following colors: royal blue, blue, yellow-green, and amber. The corresponding emission spectra may include 380-500 nm for royal blue light, 400-520 nm for blue light, 400-710 nm for yellow-green light and 475-800 nm for amber light. In addition, at least one LED die may be configured to emit light at a wavelength greater than 850 nm. Regardless of the emission spectrum, the at least one LED 20 may be dimmable. A user may be able to select a custom wavelength for light emission from the at least one LED 20.

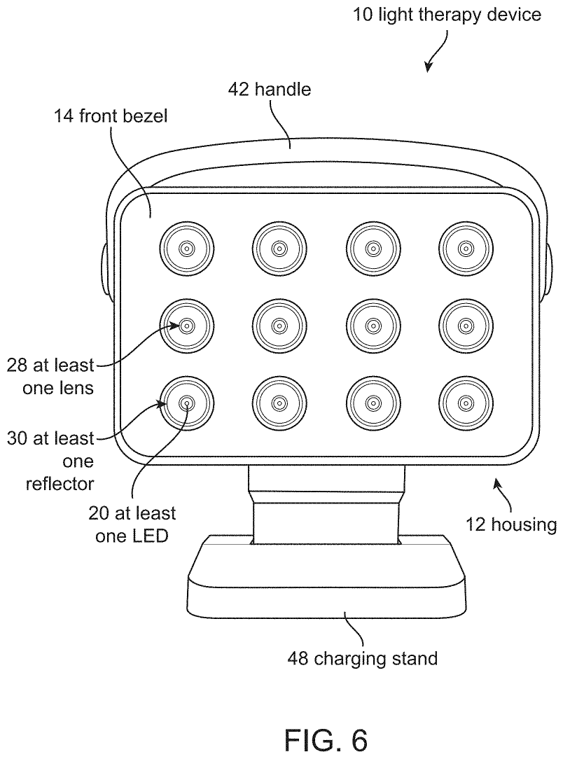

Turning now to FIG. 6, a front view of the device 10 is shown. As previously discussed, the device 10 may include a housing 12 comprising a front bezel 14, a handle 42, and may be coupled to a charging stand 48. FIG. 6 also includes the at least one lens 28, the at least one reflector 30, and the at least one LED 20. As discussed with reference to FIGS. 2 and 3, the at least one lens 28 and at least one reflector 30 may be integrated into the front bezel 14. The at least one LED 20 may be arranged in three rows of four for a total of twelve LEDs, as demonstrated in FIG. 6. The at least one LED may be arranged in four rows of three, six rows or two, or any other suitable arrangement to substantially evenly space the twelve LEDs and distribute them across the front bezel 14. In some embodiments, the at least one LED 20 comprises more than twelve LEDs. The at least one LED 20 may comprise fewer than twelve LEDs. The LEDs may be arranged in a shape other than even lines; for example, in a single circle or concentric circles. Any arrangement of LEDs may be suitable to provide light therapy to a user of the device 10.

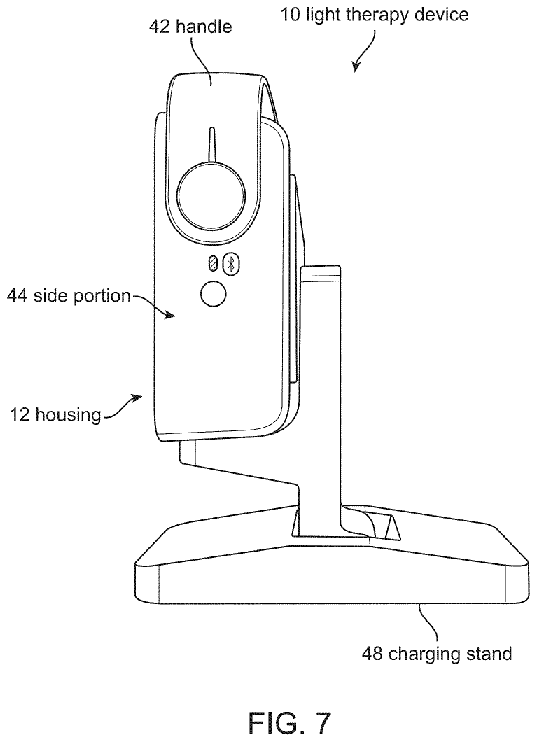

The handle 42 may be configured to couple to at least one side portion 44 of the housing 12, as shown in FIG. 7. In some embodiments, the handle 42 is detachably coupled to the at least one side portion 44. For example, as illustrated in FIG. 7, the housing 12 may comprise a substantially round protrusion located on the side portion 44 and the handle 42 may comprise a narrow opening (e.g., a slit) configured to flex open in order to receive the protrusion. The protrusion may define any shape and is not limited to a round protrusion. The narrow opening may define a height of about 19 mm and a width, when not flexed, of about 1 mm. The handle 42 may be coupled to the housing 12 such that it is configured to rotate, slide, or otherwise adjust position. Such a flexible configuration may enable any number of users to adjust the handle 42 for comfortable holding and/or transport of the device 10.

In some embodiments, the handle 42 is comprised of a flexible material such as rubber. The handle 42 may comprise an inflexible (or rigid) material. The handle 42 may comprise a combination of materials. For example, an area around the narrow opening configured to receive the protrusion to thereby couple the handle 42 to the housing 12 may comprise a flexible material while the rest of the handle 42 comprises an inflexible material. In some embodiments, the handle 42 comprises a textured surface. The handle 42 may comprise a substantially smooth surface. Example dimensions of the handle 42 are as follows: about 68 mm height, about 3 mm thickness, about 153 mm outer length, and about 27 mm width. Any of the dimensions of the handle 42 may be greater or lesser than the listed example dimensions. The handle 42 may couple to any portion of the housing 12, including, but not limited to, the front bezel 14, the back portion 16, a corner portion, and a top portion.

In many embodiments, the device 10 includes at least one user control located on the housing 12. The at least one user control may be located on a side portion 44 of the housing 12. The at least one user control may comprise at least one button and at least one indicator, such as a LED. The at least one user control may be at least one of operatively and electrically coupled to the control board 34. In some embodiments, the at least one user control comprises at least one of a power button, a status LED, a pairing button, and a pairing LED. The power button and status LED may be located on one side portion 44 of the housing 12, and the pairing button and pairing LED may be located on the other side portion 44 of the housing 12. The at least one user control shown in FIG. 7 may comprise the pairing button and pairing LED. In some embodiments, the status LED comprises a tri-color LED configured to emit, for example, blue, yellow, and green light. The pairing LED may comprise a dual-color LED configured to emit, for example, blue and green light. In some embodiments, the pairing LED comprises a single color LED. The status LED may comprise a single color, dual-color, tri-color, quad-color, etc., LED. At least one of the status LED and pairing LED may be configured to emit light in a solid color, a flashing color, or any other pattern, including patterns of varying speed.

In some embodiments, the power button is configured to turn the device 10 on, turn the device 10 off, and reset the device 10. The pairing button may be used to enable and disable an airplane mode, as well as to pair the device 10 to a remote computing device, via a Bluetooth connection, for use with a mobile application. The Bluetooth connection may comprise a Bluetooth 5.0 connection. In many embodiments, the device 10 is configured to retain Bluetooth pairing to a remote computing device after a power on/off cycle. At least one of the at least one user control and the remote computing device may be configured to initiate at least one operational mode of the device 10. In some embodiments, the at least one operational mode comprises at least one of a Recovery Plus Mode, an Ambient Mode, and an Alarm Clock Mode.

When the device 10 is arranged and configured to operate in the Recovery Plus Mode, the at least one LED 20 may be configured to emit pulsed infrared light. In many embodiments, the at least one LED 20 is configured to emit substantially continuous red light in the Recovery Plus Mode. The pulsed infrared light may be emitted at a single frequency. In some embodiments, the infrared light is pulsed at varying frequencies throughout the duration of the Recovery Plus Mode. “Varying” frequencies may comprise frequencies between 0 and 100 Hz, emitted at different duty cycle percentages. For example, the Recovery Plus Mode may include pulsing infrared light at 25 Hz and at 50% brightness for two minutes, increasing the frequency to 50 Hz at 75% brightness for two minutes, then reducing the frequency to 35 Hz and 40% brightness for two minutes. The Recovery Plus Mode may comprise any combination of frequency and brightness per duty cycle. The infrared light may be pulsed for a certain amount of time; for example, the infrared light may be emitted for two seconds every five seconds. The infrared light may be pulsed for a shorter or longer duration than two seconds. In some embodiments, the Recovery Plus Mode comprises pulsing red light and substantially continuous infrared light. The Recovery Plus Mode may include pulsed or continuous emission of infrared and/or near infrared light. The duration of a treatment session using Recovery Plus Mode may be selected by a user of the device 10 via at least one of the mobile application and the at least one user control. The Recovery Plus Mode may help facilitate cellular recovery by encouraging the removal of waste products from cells during a “quench period” in the cellular recovery process. The device 10 may be configured to automatically shut off at the end of a Recovery Plus Mode session. A user may manually shut off the device 10.

When the device 10 is arranged and configured to operate in the Ambient Mode, the at least one LED 20 may be configured to emit ambient light. In many embodiments, the at least one LED 20 is configured to emit substantially continuous red light in the Ambient Mode. The Ambient Mode may be configured to operate for any duration selected by a user; for example, ten minutes, twenty minutes, thirty minutes, sixty minutes, etc. The device 10 may be configured to automatically shut off after the preselected duration for Ambient Mode. A user may manually shut off the device 10. In many embodiments, the Ambient Mode is configured such that the at least one LED 20 emits substantially continuous red light at 50% of maximum brightness. The user may select a desired brightness level for Ambient Mode. Though used for “ambient” light purposes, during the Ambient Mode, the user may experience any of the various benefits provided by exposure to red light.

When the device 10 is arranged and configured to operate in the Alarm Clock Mode, the at least one LED 20 may be configured to emit ambient light upon at least one of a predetermined condition and at a predetermined time. It should be noted that the ambient light emitted in the Alarm Clock Mode may comprise the same light emitted in the Ambient Mode—substantially continuous red light emitted at 50% brightness. The ambient light emitted in the Alarm Clock Mode may include a ramp-up sequence before reaching a predetermined brightness level (e.g., 50%), wherein the at least one LED 20 is configured to emit light of increasing intensity over a predetermined period of time. For example, the at least one LED 20 may be configured such that, upon (or before) the predetermined condition and/or at (or before) the predetermined time, the at least one LED 20 is configured to turn on and emit red light at 10% brightness with a gradual increase to 50%. The user may select the beginning brightness and the duration of the ramp-up sequence, as well as the peak brightness level.

For example, the user may program the Alarm Clock Mode such that the device 10 is configured to emit red light at 50% brightness at 6:30 AM every morning, Monday-Friday. Beginning at 6:20 AM on Monday-Friday, the at least one LED 20 may be configured to emit red light at 10% brightness. The at least one LED 20 may be configured to gradually increase the brightness such that at 6:25 AM, the at least one LED 20 emits red light at 25% brightness. The at least one LED 20 may continue gradually increasing the brightness over the next five minutes such that at 6:30 AM, the red light is emitted at 50% brightness. A user may forgo the ramp-up sequence and instead configure the device 10 to turn on and immediately emit red light at the predetermined brightness at the predetermined time.

In some embodiments, the Alarm Clock Mode is configured such that the at least one LED 20 emits ambient light upon a predetermined condition. The predetermined condition may comprise a variety of conditions. For example, a remote computing device of the user may be communicatively coupled to the device 10 such that a mobile application on the remote computing device is configured to trigger the at least one LED 20. In some embodiments, the mobile application comprises a sleep tracking application configured to trigger the at least one LED 20 after a predetermined amount of sleep. The sleep tracking application may be configured to trigger the at least one LED 20 upon detection that the user is in a “light” phase of sleep, which may make it easier and more pleasant for the user to wake up.

As another example, the mobile application may be communicatively coupled to a smart device of the user's home, such as a coffee maker. In some embodiments, the mobile application is configured to trigger the at least one LED 20 upon completion of brewing at least one of a pot and a cup of coffee. The mobile application may be configured to trigger the at least one LED 20 upon starting to brew at least one of a pot and a cup of coffee. The mobile application may be coupled to another smart device, such as a smart thermostat. In some embodiments, the mobile application is configured to trigger the at least one LED 20 when the smart thermostat reaches a predetermined temperature.

“Triggering” the at least one LED 20 may comprise starting a ramp-up sequence. In some embodiments, “triggering” the at least one LED 20 comprises immediately emitting ambient light at the predetermined brightness (e.g., 50%). The Alarm Clock Mode may be configured to automatically shut off the at least one LED 20 after a predetermined amount of time. For example, the at least one LED 20 may be configured to shut off thirty minutes after at least one of the predetermined time and the occurrence of the predetermined condition. The user may manually turn off the device 10.

As previously mentioned, the at least one LED 20 may comprise a dimmable LED. A dimming feature may be included in any or all of the Recovery Plus Mode, the Ambient Mode, and the Alarm Clock Mode. The dimming feature may also be integrated into a light therapy session using the device 10. In some embodiments, regardless of the Mode, the at least one LED 20 may ramp-up from off to full (or a predetermined) brightness in a ten second period. When the device 10 is turned off and/or at the end of a treatment session, the at least one LED 20 may ramp-down from the existing brightness level to off in a ten second period. The ramp-up and/or ramp-down period may comprise more or fewer than ten seconds.

In some embodiments, at least one of the Recovery Plus Mode, the Ambient Mode, and the Alarm Clock Mode includes a beeper function. The device 10 may be configured to emit a sound (e.g., “beep”) as an indication of progress of a session using at least one of the stated Modes. For example, during the Recovery Plus Mode, the device 10 may be configured to beep at the beginning of a session, when two minutes remain in the session, and at the conclusion of the session. The device 10 may be configured to emit a sound as an indication of progress in a “regular” session that does not use one of the stated modes. Beeper functionality, including the volume of the sound emitted, the type of sound emitted (e.g., beep, chirp, chime, or the like), the number of times the sound is emitted (e.g., single beep vs. double beep), and when the sound is emitted (e.g., start of a session, halfway through a session, five minutes remaining, one minute remaining, end of session, every two minutes during a session, etc.) may be customizable based on preferences of the user. Beeper functionality may be programed via the device 10, using the at least one user control, or via a mobile application on the remote computing device. A user may choose to forgo the beeper function so that the device 10 does not emit any sound during operation.

Referring now to FIG. 8, a back view of the device 10 is shown. As previously stated, the device 10 may include a housing 12 comprising a front bezel 14 (shown in FIGS. 1, 2, and 6) configured to couple to the back portion 16. The back portion 16 may include a second handle, a grip, or a similar mechanism configured to be held by a user of the device 10. The handle, grip, or the like may be integrated into the back portion 16 (e.g., as an element molded into the back portion 16) or may comprise a separate component mechanically coupled to the back portion 16.

In many embodiments, the device 10 is configured to couple to a cradle of the charging stand 48. FIGS. 7 and 8 show that the cradle of the charging stand 48 may comprise a portion configured to couple to the back portion 16 of the device 10. In some embodiments, the device 10 is configured to restably couple (e.g., lean against) the back portion of the charging stand 48. The device 10 may be mechanically coupled to the back portion of the charging stand 48. The charging stand 48 may comprise a tiltable arm such that the position of the device 10 may be tilted to adjust the angle of light emission from the device 10. The “arm” of the charging stand 48 may be considered a portion of the stand 48 coupling the cradle to a base portion of the stand 48. In some embodiments, the cradle of the charging stand 48 is tiltable with respect to the arm of the charging stand 48. The arm of the charging stand 48 may be tiltable with respect to the base of the stand 48. The arm and the cradle may be tiltable with respect to one another.

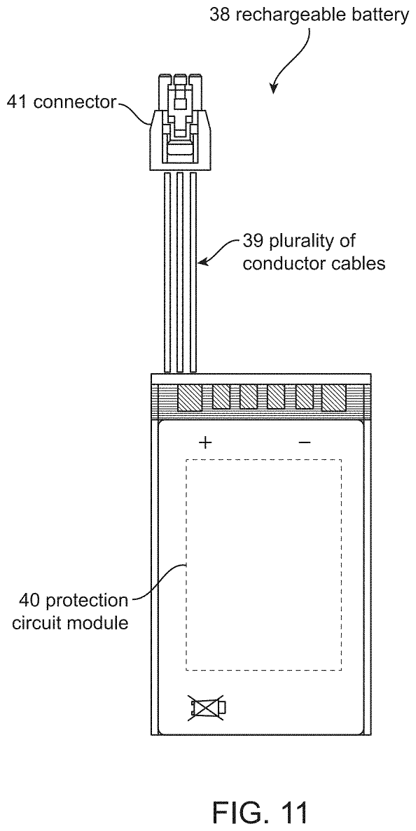

In many embodiments, the device 10 is at least one of mechanically and electrically coupled to the charging stand 48 via a bottom portion 49 of the housing 12, as shown in FIG. 9. More specifically, the device 10 may be configured to restably couple to the cradle of the charging stand 48 via at least a portion of the bottom portion 49 and electrically couple to the charging stand 48 via the charging contact 50a. In many embodiments, the charging contact 50a of the device 10 is configured to electrically couple to a charging contact 50b (shown in FIG. 10) located on the cradle of the charging stand 48. The electrical coupling may be configured to charge the rechargeable battery 38 (shown in FIG. 11) of the device 10. In many embodiments, the device 10 is configured to charge the rechargeable battery 38 and operate (i.e., emit light from the at least one LED 20) substantially simultaneously.

The device 10 may be configured for non-stand charging. Stated differently, in some embodiments, the rechargeable battery 38 of the device 10 is configured to receive a charge from a source other than the charging stand 48. In some embodiments, the bottom portion 49 comprises at least one input port configured to electrically couple to a charging cable. The charging cable may be configured to provide 9V at 2 Amps of DC input power. In some embodiments, the charging cable comprises a portion of a 9V, 2 A, AC-DC power adapter. The power adapter may comprise a medical grade (Class II) power adapter. As such, the device 10 may be considered a Class II medical device. The power adapter may include at least one of the following features: an AC input voltage of 100-240V and 50-60 Hz; a DC line cable length of about 120 cm, and a DC jack measuring about 5.5 mm with a positive center pin measuring about 2 mm. The power adapter may meet UL/cUL (Underwriter Laboratories, USA and Canada), FCC (Federal Communications Commission), TÜV (Technischer Überwachungsverein), and EMC (Electromagnetic Compatibility) safety approvals. In some embodiments, the charging cable is configured to couple to the charging stand 48 in order to provide power to the stand 48.

The bottom portion 49 may include an additional port, such as a mini-USB port, configured to couple the device 10 to a remote computing device in order to receive firmware and/or software updates. The mini-USB, or similar port, may be configured to transfer data from the device 10 to a remote computing device. In some embodiments, the data comprises light therapy session data including the number of sessions, duration of sessions, light emission spectra of sessions, and sessions including at least one of Recovery Plus Mode, Ambient Mode, and Alarm Clock Mode. In some embodiments, the device 10 is configured to store the light therapy session data until the data is retrieved via the mini-USB or similar port. The session data may be stored on a memory chip coupled to the PCBA 18. In some embodiments, the device 10 is configured to share session data with a remote computing device via a wireless connection, such as a Bluetooth connection. The device 10 may be communicatively coupled to the remote computing device via any other suitable wireless connection, such as via at least one of a cellular and WiFi network. The session data may remain on the device 10 even after the data is shared with the remote computing device.

FIG. 10 shows a perspective view of the charging stand 48. As previously mentioned, the charging stand 48 may include a cradle portion configured to hold the device 10. As such, the charging stand 48 may function as both a charging dock and a stand for the device 10. The charging stand 48 may be configured to restably couple to a variety of surfaces including, but not limited to, a table, a countertop, a desk, a bed, a chair, and a floor surface. As discussed with reference to FIG. 9, the charging stand 48 may include a charging contact 50b configured to electrically couple to the charging contact 50a of the device 10, thereby providing power to the rechargeable battery 38 of the device 10. In some embodiments, at least one of the charging contact 50a and the charging contact 50b comprises a spring connector. At least one of the charging contact 50a and the charging contact 50b may comprise magnetic material such that the charging contact 50a and the charging contact 50b are configured to magnetically couple. Magnetic coupling may enable the device 10 to retain a charging position on the charging stand 48 and reduce the chance of severing the charging connection after a small movement, bump, etc., of the device 10 and/or the charging stand 48.

Similar to the charging cable, the charging stand 48 may be configured to provide 9V of power to the device 10 in order to charge the rechargeable battery 38. In some embodiments, the battery 38 is configured to receive 1.5 A of charging current. The battery 38 may be configured to receive 0.5 A of charging current. In some embodiments, the battery 38 is configured to receive a charging current between 0.5 A and 1.5 A. The battery 38 may be configured to receive charging current above 1.5 A. The battery 38 may be configured to receive charging current below 0.5 A. In some embodiments, the status LED discussed with reference to FIG. 7 is configured to illuminate different colors based on a charge status of the rechargeable battery 38. For example, the status LED may be configured to illuminate green when the battery 38 is fully charged, blue while the battery 38 is charging, and yellow when the battery 38 is low on charge. In some embodiments, when the battery 38 is “dead” or substantially empty, the status LED does not illuminate until placed on the charging stand 48 (or connected to a charging cable), at which point the status LED illuminates blue.

FIG. 11 shows a perspective view of the rechargeable battery 38. In many embodiments, the rechargeable battery 38 comprises a lithium-ion (“Li-ion”) polymer battery. The rechargeable battery 38 may comprise any other suitable type of rechargeable battery. In many embodiments, the rechargeable battery 38 includes a protection circuit module 40 configured to protect the rechargeable battery 38 from damage, such as damage that can occur from over-charging or over-discharging. The damage that can occur may be severe, including explosion of the battery. In many embodiments, the rechargeable battery 38 complies with IEC 62133-2:2017 safety requirements (for secondary cells and batteries containing alkaline or other-non-acid electrolytes). The rechargeable battery 38 may also comply with all safety requirements for Lithium cells used in portable applications.

In some embodiments, the rechargeable battery 38 also includes a plurality of conductor cables 39 and a connector 41, as shown in FIG. 11. The plurality of conductor cables 39 may comprise three conductor cables. The connector 41 may comprise a Molex-105307-1203 with a current capacity of 6 A per pin, or similar connector. In some embodiments, the connector 41 is configured to couple to the charging contact 50a of the device 10 to thereby receive power from the charging contact 50a, via a docking printed circuit board assembly. In some embodiments, the docking printed circuit board is electrically coupled to the charging contact 50a and the rechargeable battery 38. The plurality of conductor cables 39 may be configured to transport power from the connector 41 to the body of the rechargeable battery 38. The rechargeable battery 38 may include the following technical parameters: battery power—15.73 W, battery voltage—4V, battery current—3.93 A. It should be noted that the listed parameters may be representative of only one embodiment of the rechargeable battery 38. In some embodiments, the device 10 includes a plurality of rechargeable batteries. The device 10 may include a single rechargeable battery 38.

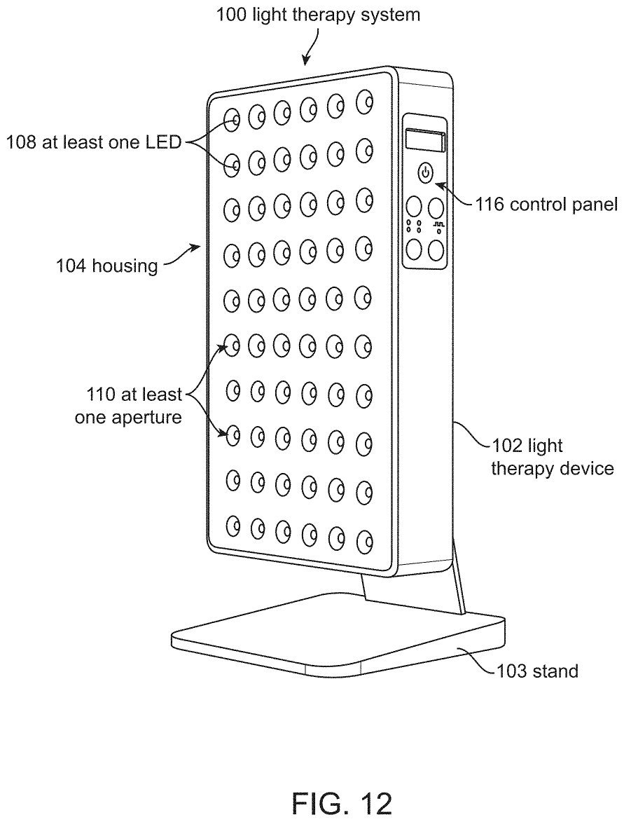

FIG. 12 shows a light therapy system 100, according to some embodiments. The system 100 may include a light therapy device 102 coupled to a stand 103. In many embodiments, the light therapy device 102 comprises a housing 104, at least one LED 108, at least one aperture 110, and a control panel 116, as shown in FIG. 12. Various elements of the light therapy device 102 may be similar to some elements of the light therapy device 10 discussed with reference to FIGS. 1-11. For example, like the at least one aperture 22, the at least one aperture 110 may comprise at least one lens and/or at least one reflector configured to be positioned over the at least one LED 108. In many embodiments, the at least one lens is configured to cover at least a portion of the at least one aperture 110 such that light travels from at least one LED 108 of the device 102 through the at least one lens. The at least one lens may have a reflection angle of about twenty degrees. The at least one reflector may be configured to surround at least a portion of the at least one LED 108 to thereby reflect the light emitted from the at least one LED 108. In some embodiments, an interior portion of the at least one reflector is coated with a reflective surface coating. The at least one reflector may be configured to concentrate the light emitted from the at least one LED 108 and direct the concentrated light toward the at least one lens.

In addition, like the at least one LED 20, the at least one LED 108 may comprise a multi-die chip LED. Each multi-die chip LED may comprise four LED die. In some embodiments, two of the four LED die comprise LEDs configured to emit red light at a wavelength of about 660 nm. The other two LED die may comprise LEDs configured to emit infrared light at a wavelength of about 850 nm. In some embodiments, the LED die configured to emit red light are configured to emit red light at a wavelength between 655 nm and 660 nm with an irradiance of about 20 mW/cm² at 6 inches. The LED die configured to emit infrared light may be configured to emit infrared light at a wavelength between about 840 nm and 870 nm with an irradiance of about 20 mW/cm² at 6 inches. It should be noted that irradiance measurements are approximate and based on measurements taken at a room temperature of 25° C. The total unit radiant flux may be equal to or greater than 65 W. The total light output may be about 8.09 W.

In some embodiments, the multi-die chip LED comprises part number PBLB-3LQE-MJ produced by ProLight Opto, of Epistar Corp. (Taiwan). In some embodiments, the at least one LED 108 comprises at least one 4-in-1 LED in a 3030 package. Stated differently, each LED of the at least one LED 108 may define a width of 3.0 mm and a length of 3.0 mm. In some embodiments, the at least one LED 108 comprises at least one 4-in-1 LED in a 3535 package. Stated differently, each LED of the at least one LED 108 may define a width of 3.5 mm and a length of 3.5 mm. Each LED of the at least one LED 108 may define a height of about 1.8 mm. The red and infrared LEDs may define a cross pattern. In some embodiments, LEDs configured to emit the light at the same wavelength are located side-by-side or top-to-bottom.

In some embodiments, the at least one LED 108 is configured to emit light at wavelengths other than 660 nm and 850 nm. For example, at least one die in the multi-die chip LED may be configured to emit light at any of the following colors: royal blue, blue, yellow-green, and amber. The corresponding emission spectra may include 380-500 nm for royal blue light, 400-520 nm for blue light, 400-710 nm for yellow-green light and 475-800 nm for amber light. In addition, at least one LED die may be configured to emit light at a wavelength greater than 850 nm. Regardless of the emission spectrum, the at least one LED 108 may be dimmable. A user may be able to select a custom wavelength for light emission from the at least one LED 108.

In many embodiments, both the devices 10, 102 are configured to provide at least one of red and infrared light therapy. It should be noted that the term “infrared light therapy”, as applied to the device 10 and/or the device 102, may include emission of infrared and/or near infrared light.

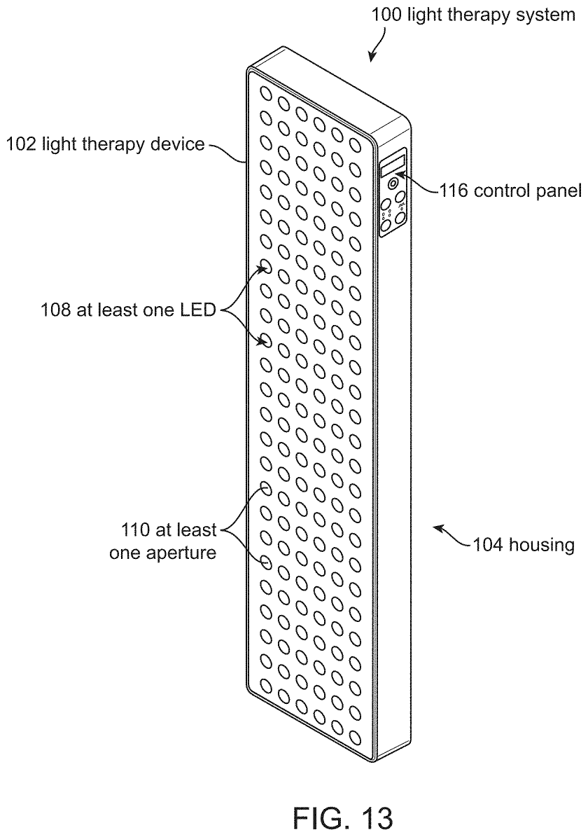

FIG. 13 shows another embodiment of the light therapy system 100, including the light therapy device 102 comprising the housing 104, the at least one LED 108, the at least one aperture 110, and the control panel 116. As demonstrated by FIGS. 12 and 13, different embodiments of the device 102 may comprise light devices of different sizes. In some embodiments, the device 102 shown in FIG. 13 is configured to couple to a door, a wall, or a stand other than the stand 103 shown in FIG. 12. For example, the device 102 shown in FIG. 13 may be configured to couple to any of the components disclosed in U.S. Non-Provisional patent application Ser. No. 17/027,338, filed Sep. 21, 2020, entitled PHOTOBIOMODULATION THERAPY DEVICE ACCESSORIES and produced by Joovv, Inc. of Delaware, USA. Different embodiments of the device 102 may comprise different numbers of LEDs in the at least one LED 108. For example, the device 102 may comprise sixty LEDs in the at least one LED 108, as shown in FIG. 12. The device 102 may comprise one hundred fifty LEDs in the at least one LED 108, as shown in FIG. 13. Of course, the device 102 may comprise any number of LEDs in the at least one LED 108, and is in no way limited to embodiments comprising exactly sixty or exactly one hundred fifty LEDs.

In many embodiments, the device 102 meets the requirements for an Ingress Protection Code rating of 21, also known as an IP21 rating. An IP21 rating may indicate that the device 102 is “Protected from touch by fingers and objects greater than 12 millimeters” and “Protected from condensation,” as defined by the International Electrotechnical Commission. Unlike the device 10, the device 102 may not be substantially waterproof.

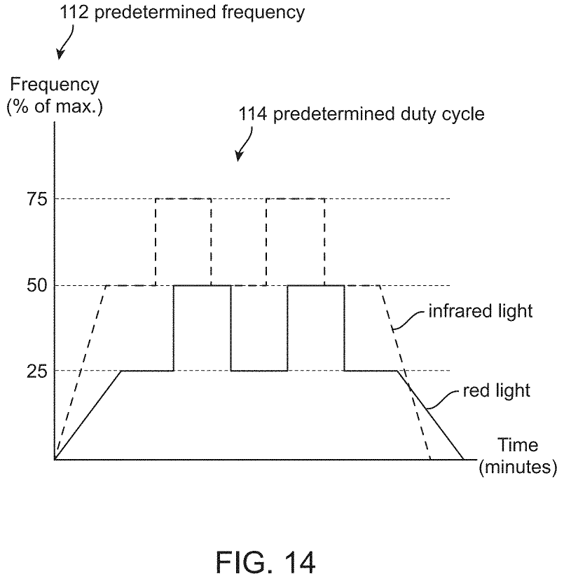

FIG. 14 shows an example graph of a predetermined duty cycle 114. In many embodiments, the at least one LED 108 is arranged and configured to pulse at a predetermined frequency 112 and a predetermined duty cycle 114. As shown in FIG. 14, the at least one LED 108 may be configured to emit infrared light at one predetermined duty cycle 114 and red light at another, different, predetermined duty cycle 114. For example, in a given session time, the at least one LED 108 may be configured to ramp-up emission of infrared light to a 50% frequency for a period of time, increase to a 75% frequency for a period of time, decrease back to 50% for a period of time, increase back to 75% for a period of time, decrease back to 50% for a final time, then ramp-down to zero emission at the end of the session. The amount of time infrared light is emitted at each predetermined frequency 112 may be substantially the same throughout the duration of the session. The amount of time may vary during the course of the session.

For red light therapy, the at least one LED 108 may be configured to ramp-up to 25% frequency for a period of time, increase to 50% frequency for a period of time, decrease back to 25% for a period of time, increase back to 50% for a period of time, decrease back to 25% for a final time, then ramp-down to zero emission at the end of the session. As with the infrared emission, the amount of time red light is emitted at each predetermined frequency 112 may be substantially the same throughout the duration of the session. The amount of time may vary during the course of the session.

The predetermined duty cycle 114 may comprise at least one predetermined frequency 112 not shown in FIG. 14. In some embodiments, red light is configured to be emitted at a higher frequency than infrared light. Red and infrared light may be emitted at the same frequency. Infrared light may be emitted at a higher frequency than red light, as illustrated in FIG. 14. In some embodiments, the predetermined duty cycle 114 comprises steady emission (e.g., constant emission at a single predetermined frequency 112) of, for example, red light, and emission of infrared light at varying frequencies. The predetermined duty cycle 114 may comprise steady emission of infrared light and emission of red light at varying frequencies. In some embodiments, the predetermined duty cycle 114 comprises emission of only red light or infrared light. Varying the predetermined frequency 112 for a predetermined duty cycle 114 may include more than two different frequencies. For example, the predetermined duty cycle 114 may include emission of red light at 10%, then 70%, then 35%, then 95%, then 100%, then 25%, then 20%, then 60%, then zero. Any combination of frequency emissions may be implemented in the predetermined duty cycle 114.

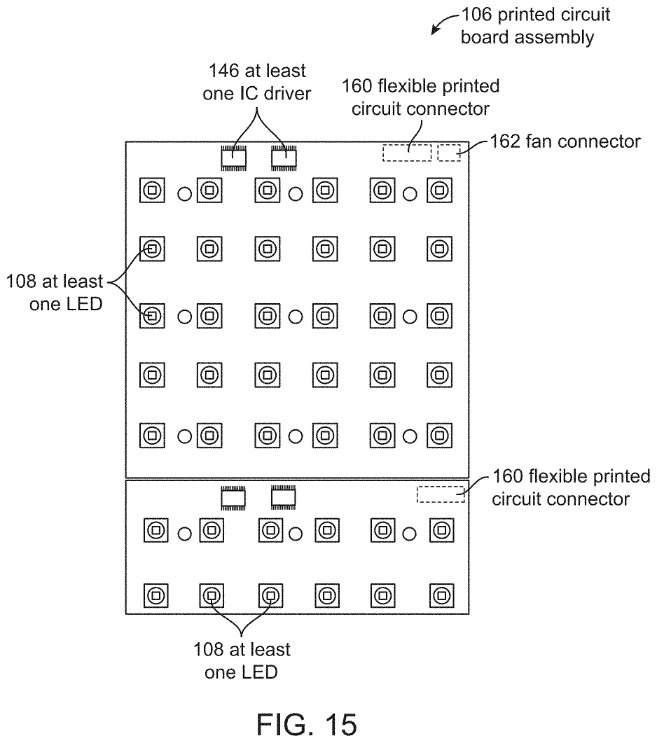

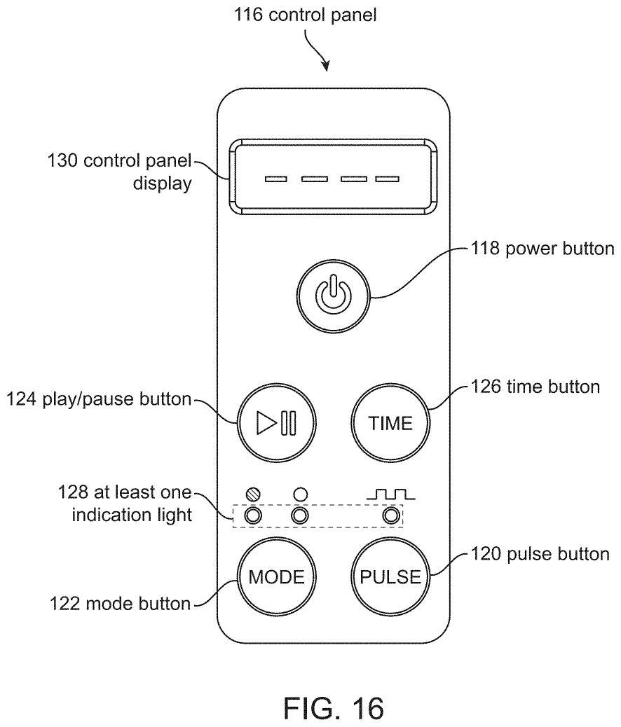

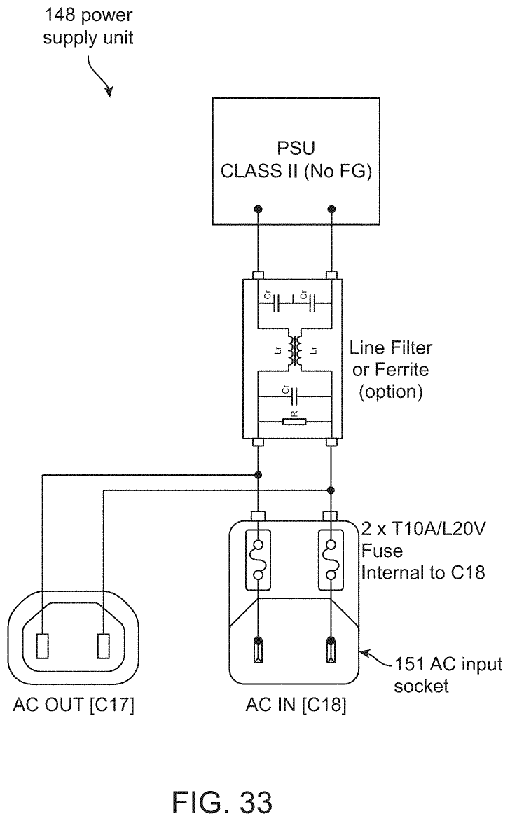

FIG. 15 shows an embodiment of a printed circuit board assembly 106 (hereafter “PCBA 106”). The PCBA 106 may be coupled to the housing 104, and the at least one LED 108 may be electrically coupled to the PCBA 106. In many embodiments, the PCBA 106 includes at least one integrated circuit (hereafter “IC”) driver 146, at least one flexible printed circuit connector 160, and at least one fan connector 162. The at least one IC driver 146 may be configured to control the brightness of the at least one LED 108. In addition, the at least one IC driver 146 may be configured to provide options for pulsing the at least one LED 108. Pulsing the at least one LED 108 will be discussed further with reference to a Pulse Mode and FIG. 16. In some embodiments, the use of the at least one IC driver 146 helps facilitate the use of a single power supply unit for the device 102. The power supply unit will be discussed in greater detail with reference to FIG. 33. The IC driver 146 may convert light from AC to DC power.

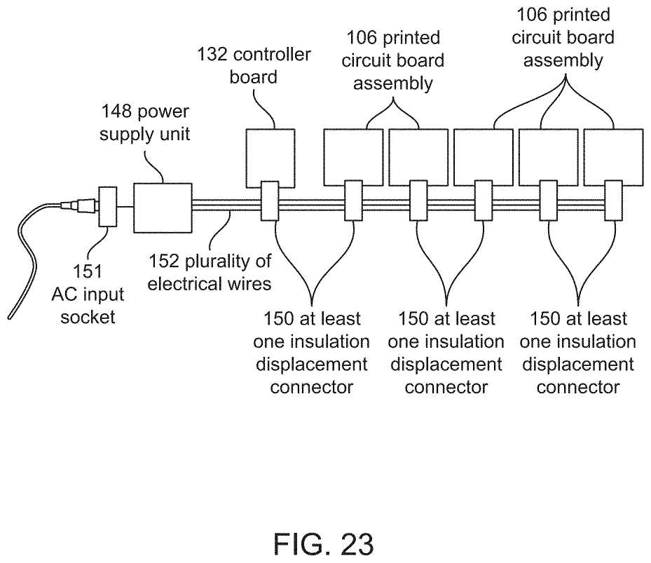

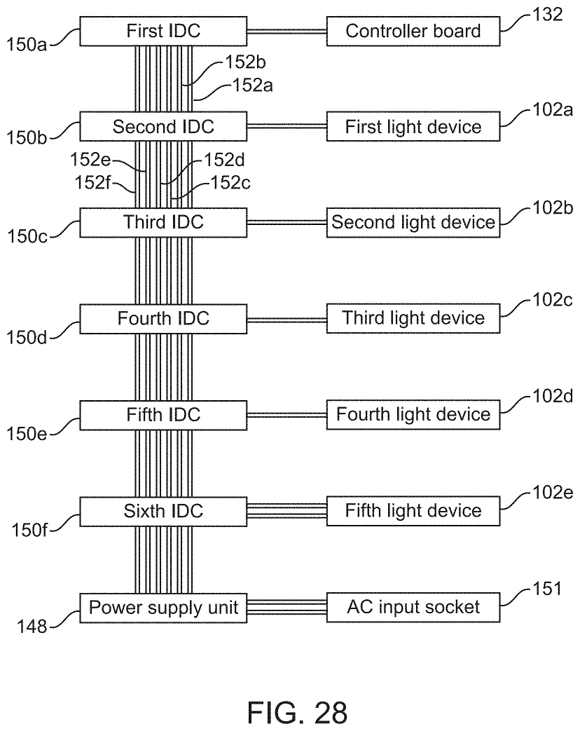

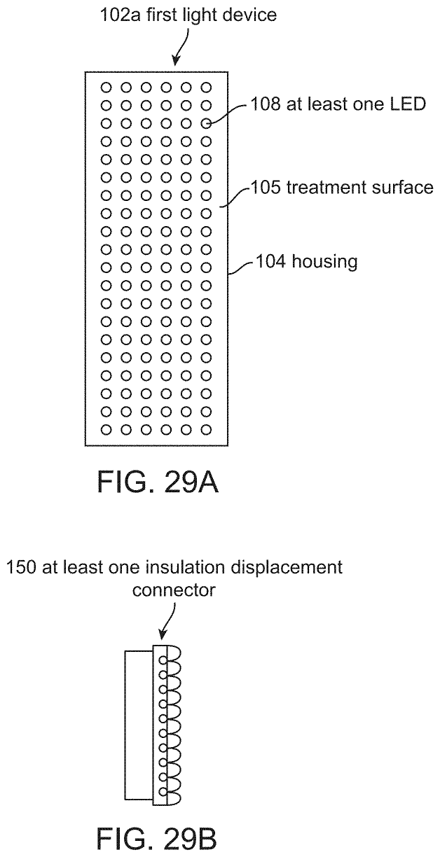

In some embodiments, the PCBA 106 is electrically coupled to a single 48V power harness. The power harness will be discussed further with reference to FIGS. 23-31. The at least one LED 108 electrically coupled to the PCBA 106 may comprise sixteen red LEDs and fourteen infrared LEDs. In some embodiments, the PCBA 106 comprises sixteen infrared LEDs and fourteen red LEDs. The at least one IC driver 146 may comprise different current setting resistor values depending on the distribution of LEDs on the PCBA 106. For example, the at least one IC driver 146 of a PCBA 106 comprising sixteen red LEDs may have a different current setting resistor value than the at least one IC driver 146 of a PCBA 106 comprising fourteen red LEDs. The at least one IC driver 146 may be configured to control the red LED loop and the infrared LED loop individually. The device 102 may comprise a plurality of PCBAs 106.

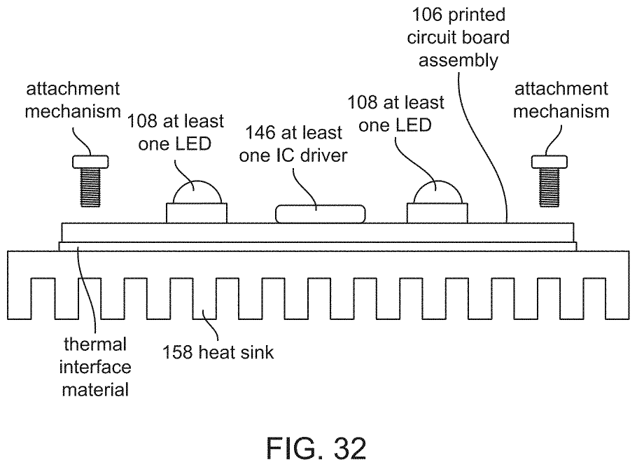

In some embodiments, the at least one flexible printed circuit connector 160 shown in FIG. 15 is used to at least one of electrically and communicatively couple one PCBA 106 to another PCBA 106 within a device 102. The fan connector 162 may be configured to at least one of electrically and communicatively couple the PCBA 106 to a fan. The fan may be configured to turn on upon receipt of a signal from the PCBA 106. The signal may indicate that the heat emitted from the PCBA 106 has exceeded a predetermined level, so that the fan may turn on to assist in dissipating the heat. The fan may turn on automatically when the device 102 is turned on. In some embodiments, the system 10 includes a plurality of fans. In addition to at least one fan, the system 10 may comprise a heat sink 158 (shown in FIG. 32) to assist in heat dissipation. In many embodiments, the at least one lens, at least one reflector, PCBA 106, and heat sink 158 comprise a single piece.

FIG. 16 shows a front view of a control panel 116. In many embodiments, the control panel 116 is coupled to the housing 104 and operatively coupled to at least one of the PCBA 106 and the at least one LED 108. The control panel 116 may be coupled to a side portion of the housing 104, as shown in FIG. 13. The control panel 116 may be coupled anywhere on the housing 104. As illustrated in FIG. 16, the control panel 116 may include a power button 118, a pulse button 120, a mode button 122, a play/pause button 124, a time button 126, at least one indication light 128, and a control panel display 130.

In some embodiments, the power button 118 is configured to operatively control power to the light therapy device 102. The power button 118 may be configured to turn the device 102 on, turn the device 102 off, and put the device 102 in a standby mode. The pulse button 120 may be configured to operatively control the pulse of the at least one LED 108. It should be noted that the term “the pulse” of the at least one LED 108 may indicate that the at least one LED 108 is arranged and configured to alternately emit light and refrain from emitting light. The play/pause button 124 may be configured to at least one of start and stop light emission from the at least one LED 108. The time button may be configured to select a time period for light emission from the at least one LED 108. The time button may also be configured to set a real time clock. The control panel display 130 may be configured to display information regarding a treatment session. The information may include, but is not limited to; a session time (elapsed or remaining), a session mode, a real time clock, and a date. The at least one indication light 128 may be configured to indicate at least one of a power status, a red light emission status, an infrared light emission status, and a pulse status.

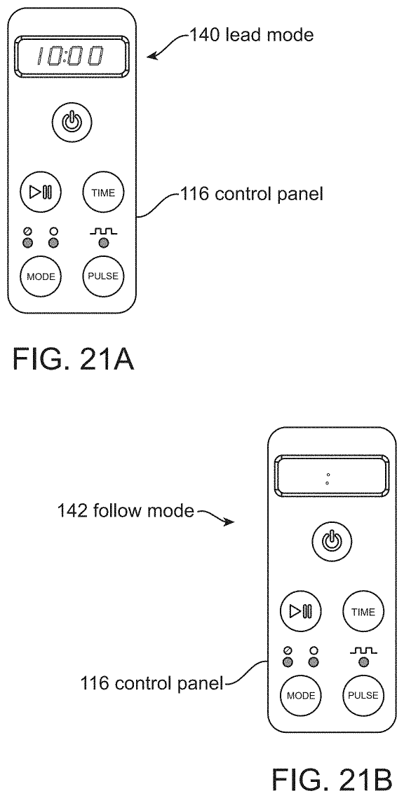

In some embodiments, the mode button 122 is configured to select a mode for light emission from the at least one LED 108. The mode for light emission may comprise at least one of a Recovery Plus Mode, an Ambient Mode, an Alarm Clock Mode, a Lead Mode 140 (shown in, and discussed with reference to, FIG. 21A), and a Follow Mode 142 (shown in, and discussed with reference to, FIG. 21B).

When the device 102 is arranged and configured to operate in the Recovery Plus Mode, the at least one LED 108 may be configured to emit pulsed infrared light. In many embodiments, the at least one LED 108 is configured to emit substantially continuous red light in the Recovery Plus Mode. The pulsed infrared light may be emitted at a single frequency. In some embodiments, the infrared light is pulsed at varying frequencies throughout the duration of the Recovery Plus Mode. “Varying” frequencies may comprise frequencies between 0 and 100 Hz, emitted at different duty cycle percentages. For example, the Recovery Plus Mode may include pulsing infrared light at 25 Hz and at 50% brightness for two minutes, increasing the frequency to 50 Hz at 75% brightness for two minutes, then reducing the frequency to 35 Hz and 40% brightness for two minutes. The Recovery Plus Mode may comprise any combination of frequency and brightness per duty cycle. The infrared light may be pulsed for a certain amount of time; for example, the infrared light may be emitted for two seconds every five seconds. The infrared light may be pulsed for a shorter or longer duration than two seconds. In some embodiments, the Recovery Plus Mode comprises pulsing red light and substantially continuous infrared light. The Recovery Plus Mode may include pulsed or continuous emission of infrared and/or near infrared light. The duration of a treatment session using Recovery Plus Mode may be selected by a user of the device 102 via at least one of the mobile application and the at least one user control. The Recovery Plus Mode may help facilitate cellular recovery by encouraging the removal of waste products from cells during a “quench period” in the cellular recovery process. The device 102 may be configured to automatically shut off at the end of a Recovery Plus Mode session. A user may manually shut off the device 102.

When the device 102 is arranged and configured to operate in the Ambient Mode, the at least one LED 108 may be configured to emit ambient light. In many embodiments, the at least one LED 108 is configured to emit substantially continuous red light in the Ambient Mode. The Ambient Mode may be configured to operate for any duration selected by a user; for example, ten minutes, twenty minutes, thirty minutes, sixty minutes, etc. The device 102 may be configured to automatically shut off following the end of the preselected duration of the Ambient Mode session. A user may manually shut off the device 102. In many embodiments, the Ambient Mode is configured such that the at least one LED 108 emits substantially continuous red light at 50% of maximum brightness. The user may select a desired brightness level for Ambient Mode. Though used for “ambient” light purposes, during the Ambient Mode, the user may experience any of the various benefits provided by exposure to red light.

When the device 102 is arranged and configured to operate in the Alarm Clock Mode, the at least one LED 108 may be configured to emit ambient light upon at least one of a predetermined condition and at a predetermined time. It should be noted that the ambient light emitted in the Alarm Clock Mode may comprise the same light emitted in the Ambient Mode—substantially continuous red light emitted at 50% brightness. The ambient light emitted in the Alarm Clock Mode may include a ramp-up sequence before reaching a predetermined brightness level (e.g., 50%), wherein the at least one LED 108 is configured to emit light of increasing intensity over a predetermined period of time. For example, the at least one LED 108 may be configured such that, upon (or before) the predetermined condition and/or at (or before) the predetermined time, the at least one LED 108 is configured to turn on and emit red light at 10% brightness with a gradual increase to 50%. The user may select the beginning brightness and the duration of the ramp-up sequence, as well as the peak brightness level.

For example, the user may program the Alarm Clock Mode such that the device 102 is configured to emit red light at 50% brightness at 6:30 AM every morning, Monday-Friday. Beginning at 6:20 AM on Monday-Friday, the at least one LED 108 may be configured to emit red light at 10% brightness. The at least one LED 108 may be configured to gradually increase the brightness such that at 6:25 AM, the at least one LED 108 emits red light at 25% brightness. The at least one LED 108 may continue gradually increasing the brightness over the next five minutes such that at 6:30 AM, the red light is emitted at 50% brightness. A user may forgo the ramp-up sequence and instead configure the device 102 to turn on and immediately emit red light at the predetermined brightness at the predetermined time.

In some embodiments, the Alarm Clock Mode is configured such that the at least one LED 108 emits ambient light upon a predetermined condition. The predetermined condition may comprise a variety of conditions. For example, a remote computing device 136 of the user may be communicatively coupled to the device 102 such that a mobile application on the remote computing device 136 is configured to trigger the at least one LED 108. In some embodiments, the mobile application comprises a sleep tracking application configured to trigger the at least one LED 108 after a predetermined amount of sleep. The sleep tracking application may be configured to trigger the at least one LED 108 upon detection that the user is in a “light” phase of sleep, which may make it easier and more pleasant for the user to wake up.

As another example, the mobile application may be communicatively coupled to a smart device of the user's home, such as a coffee maker. In some embodiments, the mobile application is configured to trigger the at least one LED 108 upon completion of brewing at least one of a pot and a cup of coffee. The mobile application may be configured to trigger the at least one LED 108 upon starting to brew at least one of a pot and a cup of coffee. The mobile application may be coupled to another smart device, such as a smart thermostat. In some embodiments, the mobile application is configured to trigger the at least one LED 108 when the smart thermostat reaches a predetermined temperature.

“Triggering” the at least one LED 108 may comprise starting a ramp-up sequence. In some embodiments, “triggering” the at least one LED 108 comprises immediately emitting ambient light at the predetermined brightness (e.g., 50%). The Alarm Clock Mode may be configured to automatically shut off the at least one LED 108 after a predetermined amount of time. For example, the at least one LED 108 may be configured to shut off thirty minutes after at least one of the predetermined time and the occurrence of the predetermined condition. The user may manually turn off the device 102.

As previously mentioned, the at least one LED 108 may comprise a dimmable LED. A dimming feature may be included in any or all of the Recovery Plus Mode, the Ambient Mode, and the Alarm Clock Mode. The dimming feature may also be integrated into a light therapy session using the device 102. In some embodiments, regardless of the Mode, the at least one LED 108 may ramp-up from off to full (or a predetermined) brightness in a ten second period. When the device 102 is turned off and/or when a treatment session is finished, the at least one LED 108 may ramp-down from the existing brightness level to off in a ten second period. The ramp-up and/or ramp-down period may comprise more or fewer than ten seconds.

In some embodiments, at least one of the Recovery Plus Mode, the Ambient Mode, and the Alarm Clock Mode includes a beeper function. The device 102 may be configured to emit a sound (e.g., “beep”) as an indication of progress of a session using at least one of the stated Modes. For example, during the Recovery Plus Mode, the device 102 may be configured to beep at the beginning of a session, when two minutes remain in the session, and at the conclusion of the session. The device 102 may be configured to emit a sound as an indication of progress in a “regular” session that does not use one of the stated modes. Beeper functionality, including the volume of the sound emitted, the type of sound emitted (e.g., beep, chirp, chime, or the like), the number of times the sound is emitted (e.g., single beep vs. double beep), and when the sound is emitted (e.g., start of a session, halfway through a session, five minutes remaining, one minute remaining, end of session, every two minutes during a session, etc.) may be customizable based on preferences of the user. Beeper functionality may be programed via the device 102, using the at least one user control, or via a mobile application on the remote computing device. A user may choose to forgo the beeper function so that the device 102 does not emit any sound during operation.

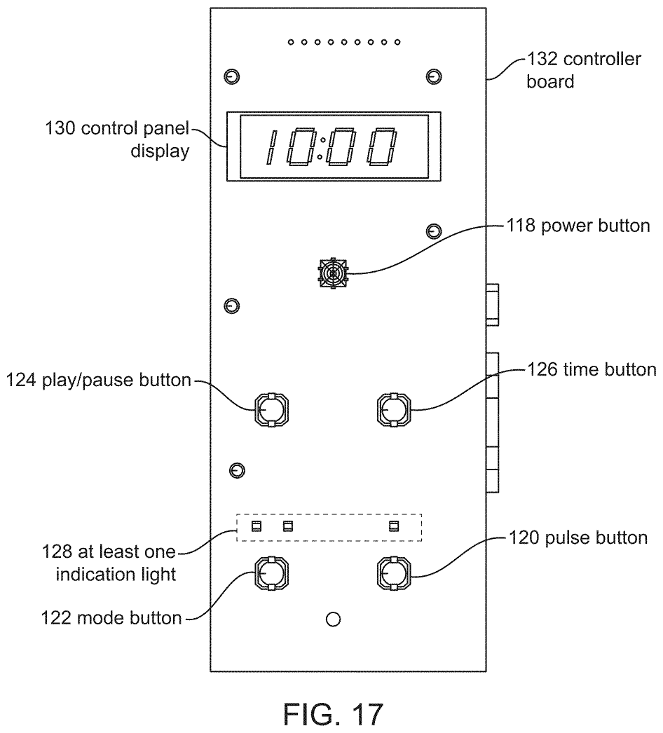

Turning now to FIG. 17, in some embodiments, the control panel 116 comprises a controller board 132 arranged and configured to operate the control panel 116. As indicated in FIG. 17, the controller board 132 may include the button inputs for each of the power button 118, time button 126, the pulse button 120, the mode button 122, and the play/pause button 124. Though not labeled in FIG. 17, the controller board 132 may also include at least one connector port configured to receive at least one cable in order to at least one of electrically and communicatively couple a controller board 132 of one device 102 to a controller board 132 of another device 102. In some embodiments, the at least one cable comprises an RJ11 cable and the at least one connector port comprises an RJ11-compatible port.

The control panel 116 may include an additional port, such as a mini-USB port, configured to couple the device 102 to a remote computing device 136 in order to receive firmware and/or software updates. The device 102 may be configured to receive firmware and/or software updates wirelessly from the remote computing device 136 via a mobile application 134. The mini-USB, or similar port, may be configured to transfer data from the device 102 to a remote computing device 136. In some embodiments, the data comprises light therapy session data including the number of sessions, duration of sessions, light emission spectra of sessions, and sessions including at least one of Recovery Plus Mode, Ambient Mode, and Alarm Clock Mode. In some embodiments, the device 102 is configured to store the light therapy session data until the data is retrieved via the mini-USB or similar port. The session data may be stored on a memory chip coupled to at least one of the controller board 132 and the PCBA 106. In some embodiments, the device 102 is configured to share session data with a remote computing device 136 via a wireless connection, such as a Bluetooth connection. The Bluetooth connection may comprise a Bluetooth 5.0 connection. The device 102 may be communicatively coupled to the remote computing device 136 via any other suitable wireless connection, such as via at least one of a cellular and WiFi network. The session data may remain on the device 102 even after the data is shared with the remote computing device 136.

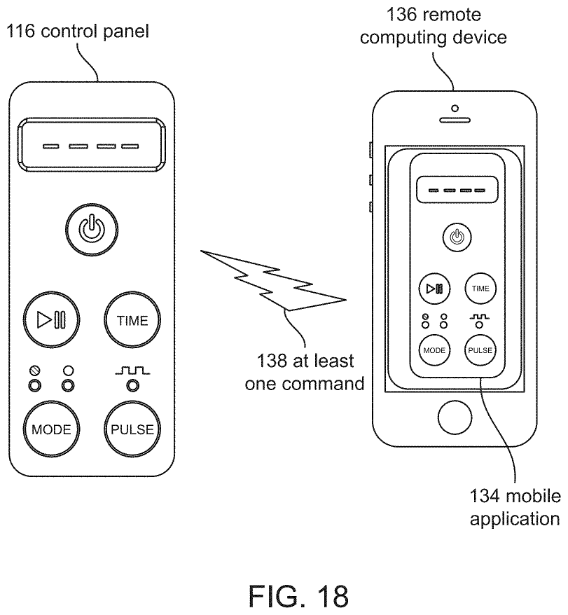

In many embodiments, the controller board 132 of the control panel 116 is communicatively coupled to a mobile application 134 on the remote computing device 136, as shown in FIG. 18. The mobile application 134 may be arranged and configured to operate the device 102 by sending at least one command 138 to the controller board 132 of the control panel 116. In some embodiments, the controller board 132 includes a Real Time Clock. The Real Time Clock may enable the device 102 to store information and understand how to tell time. In some embodiments, the Real Time Clock is useful for communicating with the mobile application 134, as the device 102 can “remember” session history and transfer information to the mobile application 134.

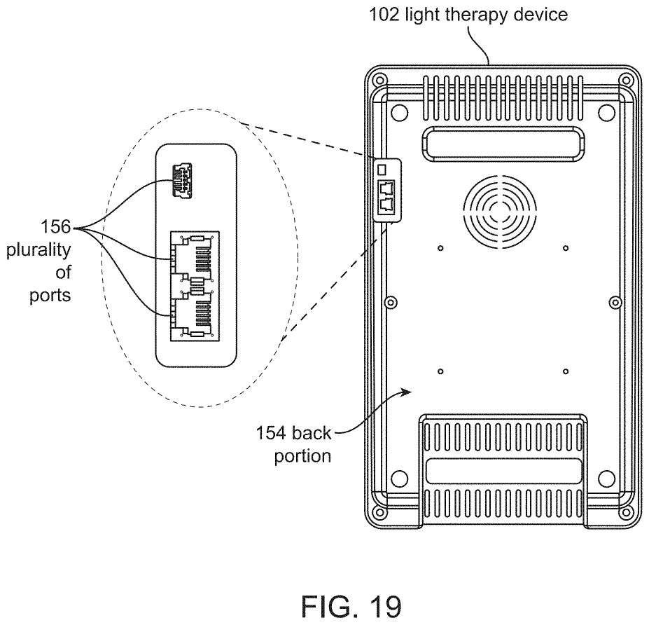

FIG. 19 shows a view of a back portion 154 of the light therapy device 102 with a zoomed-in view of a plurality of ports 156. In many embodiments, the plurality of ports 156 comprises the mini-USB (or similar) port and the dual RJ11-compatible (or similar) ports previously discussed in this disclosure. Though illustrated on the back portion 154, the plurality of ports 156 may be located on a side portion, top portion, bottom portion, or front portion of the housing 104. In some embodiments, the mini-USB port is coupled to one location of the housing 104 and the dual RJ11-compatible ports are coupled to a different location of the housing 104. The back portion 154 may also include an AC-input socket for providing power to the device 102. As previously mentioned, the device 102 may include at least one fan to assist in dissipation of the heat produced by the PCBA 106. Accordingly, the back portion 154 of the device 102 may include at least one ventilation opening in the housing 104 in order to provide air to an internal portion of the device 102. The at least one fan may comprise at least one of a traditional fan and a side blow fan. In many embodiments, the at least one fan comprises a low-noise fan.

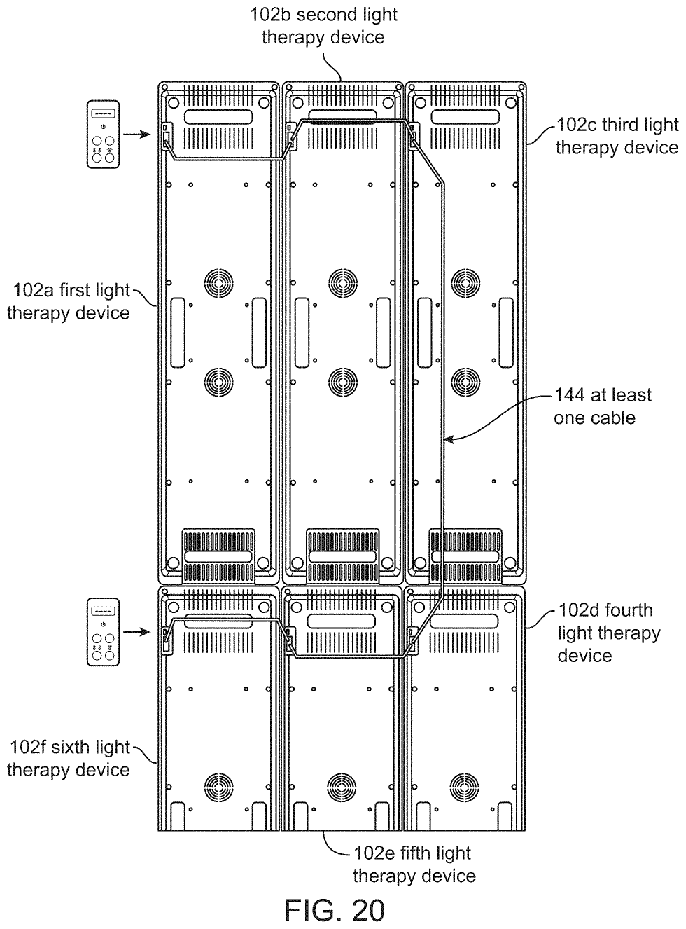

FIG. 20 shows the system 100 including six light therapy devices: a first light therapy device 102a, a second light therapy device 102b, a third light therapy device 102c, a fourth light therapy device 102d, a fifth light therapy device 102e, and a sixth light therapy device 102f. In many embodiments, the light therapy devices 102a-f are at least one of mechanically, electrically, and communicatively coupled to one another via at least one cable 144 in a “daisy-chain” configuration. The at least one cable 144 may comprise an RJ11 (or similar) cable. In some embodiments, an RJ11 cable is configured to couple two light therapy devices 102. Accordingly, in the arrangement illustrated in FIG. 20, the system 100 may require five RJ11 cables to couple the six light therapy devices 102. Though illustrated with six light therapy devices 102, it should be noted that in some embodiments, the system 100 includes fewer than six devices 102. The system may include more than six light therapy devices 102.

In many embodiments, when the system 100 includes a plurality of light therapy devices 102, a first light therapy device 102a is configured to operate in a lead mode 140 and a second (or third, fourth, etc.) light therapy device 102b (102c, 102d, etc.) is configured to operate in a follow mode 142. FIGS. 21A and 21B illustrate a control panel 116 in a lead mode 140 and a follow mode 142. In some embodiments, when the first light therapy device 102a is in the lead mode 140 and the second light therapy device 102b is in the follow mode 142, the second light therapy device 102b is configured to be controlled via the control panel 116 of the first light therapy device 102a. As such, when the first light therapy device 102a is in the lead mode 140, the control panel 116 is active, as illustrated in FIG. 21A. When the second light therapy device 102b is in the follow mode 142, the control panel 116 may be inactive, as illustrated in FIG. 21B. An inactive control panel 116 may comprise a blank control panel display 130 and non-illuminated at least one indication light 128.

In some embodiments, the first light therapy device 102a is configured to enter the lead mode 140 upon pressing the power button 118 for at least a predetermined amount of time. The second light therapy device 102b may be configured to enter the follow mode 142 upon pressing the power button 118 for at least a predetermined amount of time. In some embodiments, a button other than the power button 118 is used to configure lead and follow modes 140, 142. For example, the mode button 122 may be used to configure lead and follow modes 140, 142 in the devices 102a, 102b. A plurality of buttons on the control panel 116 may be used to configure lead and follow modes. In some embodiments, the device 102 in lead mode 140 is configured (e.g., lead mode is set) before the device 102 in follow mode 142 is configured (e.g., follow mode is set). The lead mode 140 and follow mode 142 setup may be achieved via the mobile application 134.

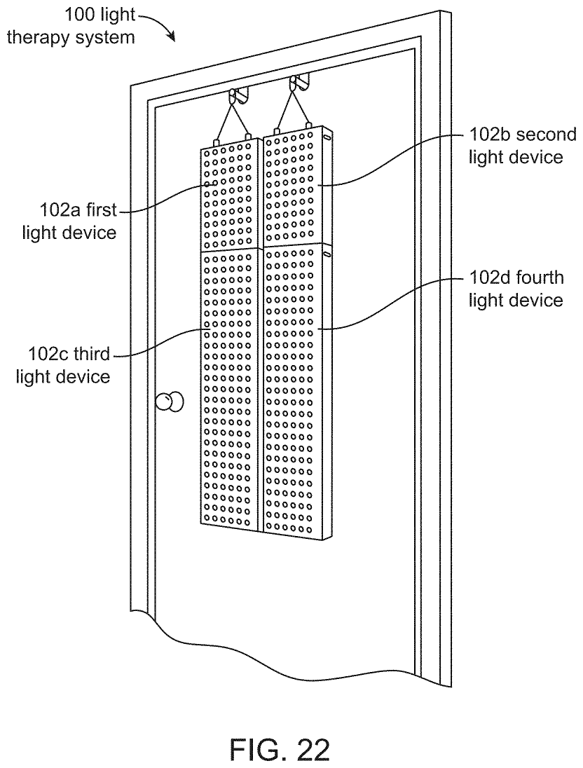

FIG. 22 shows a perspective view of the system 100, including four light therapy devices 102a, 102b, 102c, and 102d. In some embodiments, the light therapy devices 102a-d are substantially the same as the light therapy devices 102 shown in FIGS. 12 and 13. As depicted, the light therapy devices 102a, 102b, 102c, and 102d may be configured to couple to a door. The light therapy devices 102a, 102b, 102c, and 102d may be configured to couple to a wall, a mobile stand, and/or a fixed stand.

FIG. 23 shows a schematic view of the system 100, including an AC input socket 151, a power supply unit 148, a plurality of electrical wires 152, a controller board 132, at least one insulation displacement connector 150, and a plurality of PCBAs 106. As indicated, the AC input socket 151 may be coupled to the power supply unit 148. In some embodiments, the AC input socket 151 is at least one of electrically and mechanically coupled to the power supply unit 148. In some embodiments, the at least one insulation displacement connector 150 comprises at least one insulation displacement connector. The plurality of electrical wires 152 may extend from the power supply unit 148 and couple to each of the controller board 132 and the PCBAs 106 via the at least one insulation displacement connector 150. In some embodiments, the use of the at least one insulation displacement connector 150 facilitates efficiency in cable management by enabling the system 100 to use a single cable harness, where the wiring for each PCBA 106 is grouped into the harness via the at least one insulation displacement connector 150. The cable harness may be configured to get thinner the further the harness extends from the power supply unit 148. As indicated in FIG. 23, the device 102 may include five PCBAs 106. The device 102 may include fewer than five PCBAs 106. For example, the embodiment of the device 102 shown in FIG. 12 may comprise two PCBAs 106. The embodiment of the device 102 shown in FIG. 13 may comprise five PCBAs 106. In some embodiments, the device 102 includes one, three, four, or more than five PCBAs 106.

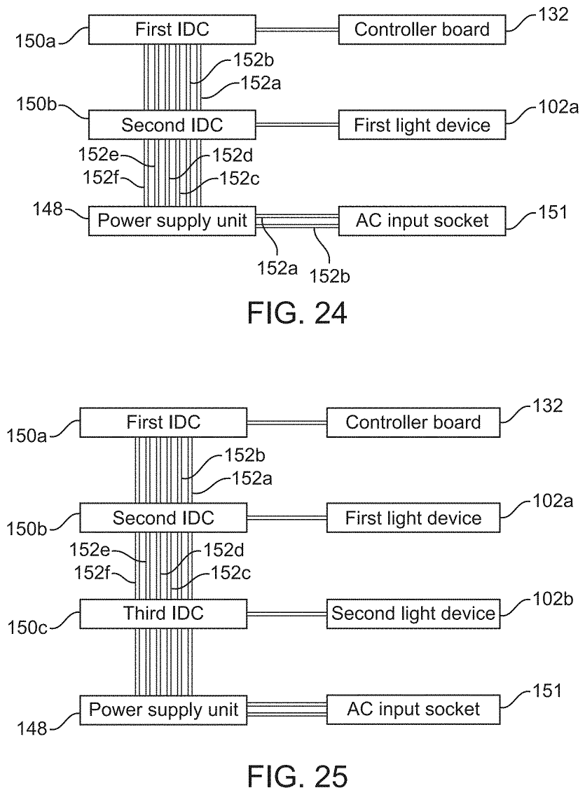

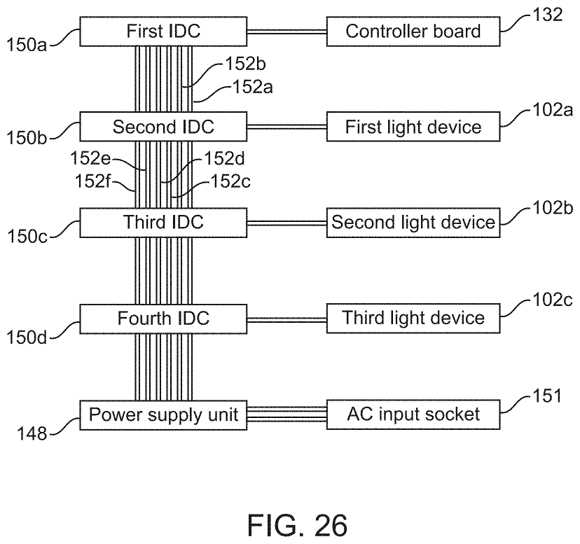

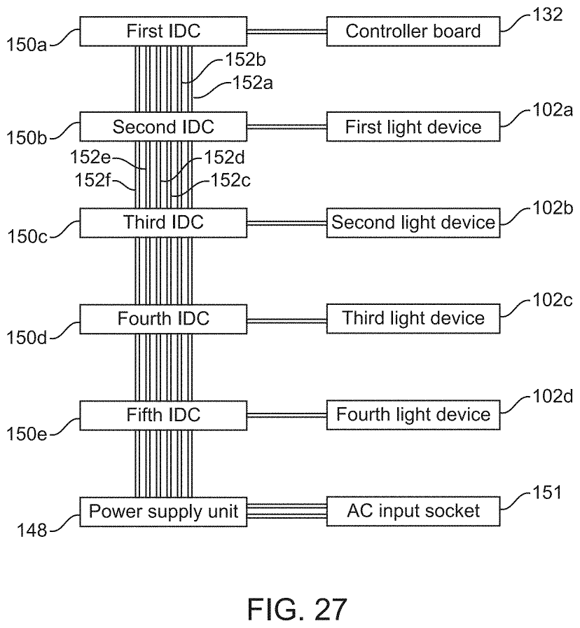

FIGS. 24-28 show electrical schematics of the system 100. FIGS. 24-28 illustrate embodiments of the system 100 with increasing numbers of light devices. For example, FIG. 24 shows an electrical schematic including a first light device 102a, FIG. 25 shows an electrical schematic including a first light device 102a and a second light device 102b, FIG. 26 shows an electrical schematic including a first light device 102a, a second light device 102b, and a third light device 102c. Each of FIGS. 24-28 also includes the AC input socket 151, the power supply unit 148, the plurality of electrical wires 152, the controller board 132, and an insulation displacement connector (represented as “IDC” in the Figures) 150 per light device 102.

In many embodiments, the controller board 132 is also coupled to an insulation displacement connector 150. The controller board 132 may be electrically coupled to a first insulation displacement connector 150a via a plurality of sharpened blades, whereby each sharpened blade of the plurality of sharpened blades penetrates the insulation surrounding each wire of the plurality of electrical wires 152 to thereby electrically and communicatively couple the first insulation displacement connector 150a to the controller board 132. The first light therapy device 102a may be at least one of electrically and communicatively coupled to a second insulation displacement connector 150b in substantially the same manner. The second light therapy device 102b may be at least one of electrically and communicatively coupled to a third insulation displacement connector 150c, and so forth for the third, fourth, and fifth light therapy devices 102c-e. In some embodiments, the system 100 includes more than five light therapy devices 102. The system 100 may comprise n+1 insulation displacement connectors 150, where “n” represents the number of light therapy devices 102.

In some embodiments, the plurality of sharpened blades comprises six sharpened blades, and the plurality of electrical wires 152 comprises six electrical wires 152a-f. The plurality of electrical wires 152 may comprise more or fewer than six electrical wires. For example, the number of electrical wires may be 4, 6, 8, 10, 12, or any other number necessary for the electrical configuration of the system 100. In many embodiments, the number of sharpened blades corresponds to the number of electrical wires in the plurality of electrical wires 152. The number of sharpened blades may be greater than the number of electrical wires in the plurality of electrical wires 152. In such an embodiments, at least one of the sharpened blades is not coupled to an electrical wire.

The plurality of electrical wires 152 is configured to transmit at least one of power and data through a first insulation displacement connector 150a, a second insulation displacement connector 150b, the AC input socket 151, and the power supply unit 148. The first and second insulation displacement connectors 150a, 150b are at least one of electrically and communicatively coupled to one another.

In many embodiments, the plurality of electrical wires 152 carries at least one of power and data through the system 100. Electrical wires 152a, 152b may carry current for power from the AC input socket 151 to the power supply unit 148, thereby electrically coupling the AC input socket 151 and power supply unit 148, as illustrated in FIG. 24. The power supply unit 148 may provide a target output voltage of 48 volts to the light therapy system. By sending data to the first light therapy device 102a, electrical wires 152c, 152d may be configured to control red light emission and electrical wires 152e, 152f may be configured to control infrared light emission.

The plurality of electrical wires 152 may be spliced at each insulation displacement connector 150a-f. The use of insulation displacement connectors 150 may be referred to as insulation displacement technology, or “IDT”. In many embodiments, these connectors 150 provide an easy and clean way for electrical coupling. All connectors 150a-f may receive at least one of power and data from a continuous wiring harness.

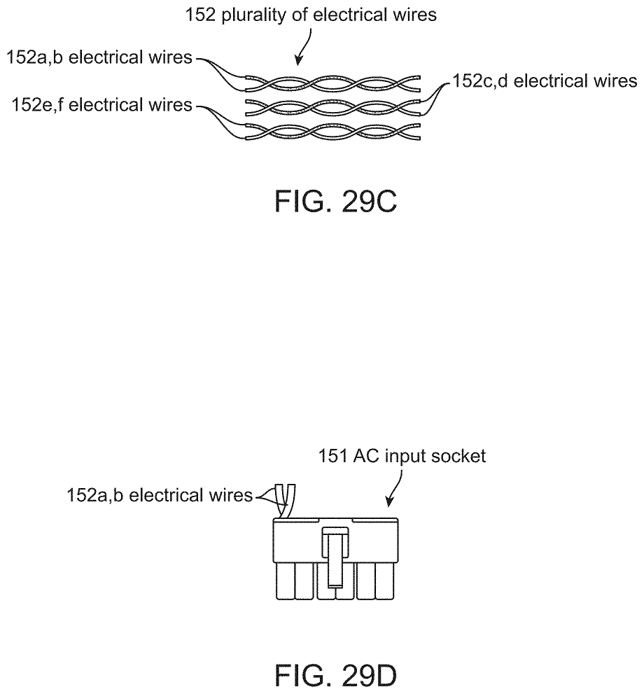

FIG. 29A illustrates the first light therapy device 102a. A treatment surface 105 on the housing 104 may include the at least one LED 108. The at least one LED 108 may be evenly distributed across each light therapy device 102, such that the light therapy device 102 includes a certain number of red LEDs and infrared LEDs. It should also be appreciated that LEDs of blue light, green light, amber light, and light of other wavelengths may be used instead of red and infrared LEDs.

As previously discussed, in some embodiments, the light therapy device 102 comprises a plurality of PCBAs 106. The at least one LED 108 of a first PCBA 106a may comprise sixteen red LEDs and fourteen infrared LEDs. A second PCBA 106b may comprise fourteen red LEDs and sixteen infrared LEDs. Like the first PCBA 106a, a third PCBA 106c may comprise sixteen red LEDs and fourteen infrared LEDs. A fourth PCBA 106d may comprise fourteen red LEDs and sixteen infrared LEDs, like the second PCBA 106b. As such, the pattern may continue with alternating numbers of red and infrared LEDs on each PCBA 106, where every other PCBA 106 comprises substantially the same proportion of each color of LEDs.

FIG. 29B illustrates an insulation displacement connector 150 configured to couple to the controller board 132 and/or light therapy devices 102a-e. In some embodiments, the insulation displacement connector 150 is configured to splice ten wires on the connection point and is configured to send at least one of power and data to the corresponding controller board 132 and/or light therapy devices 102a-e. As previously stated, the insulation displacement connector 150 may comprise any number of electrical wires, depending on the size and/or configuration of the system 100 and the number of light therapy devices 102.

FIG. 29C illustrates an example embodiment of a portion of the plurality of electrical wires 152. The plurality of electrical wires 152 may comprise six electrical wires 152a-f. In some embodiments, this number of electrical wires 152 is configured to couple to a controller board 132 and at least one light therapy device 102.

FIG. 29D illustrates an AC input socket 151, including front, top, and side views. In some embodiments, only electrical wires 152a-b carrying power may pass from the AC input socket 151 to the power supply unit 148, thereby supplying power to the light therapy system 100.

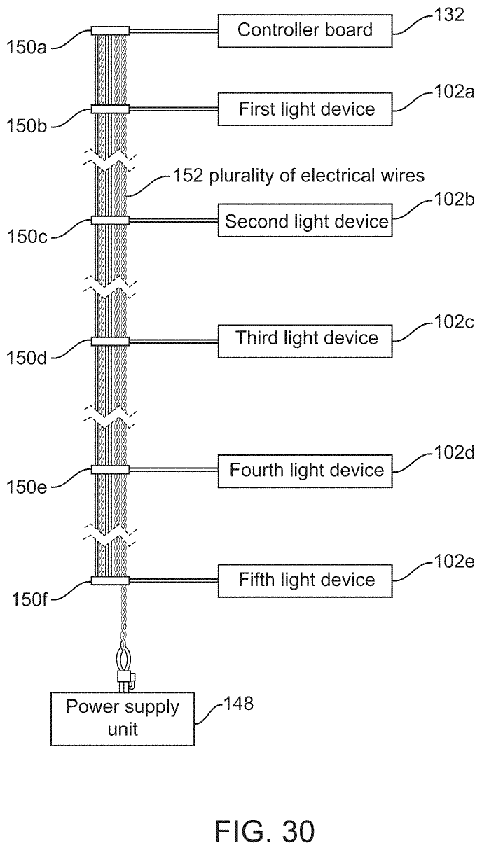

FIG. 30 illustrates an electrical schematic of the light therapy system 100. The plurality of electrical wires 152 may electrically couple to the insulation displacement connectors 150a-f, thereby providing power from the power supply unit 148 to the light therapy devices 102a-e and the controller board 132. As previously discussed, the plurality of electrical wires may be configured to carry data, as well as power, from the insulation displacement connectors 150a-f to the light therapy devices 102a-e and the controller board 132.

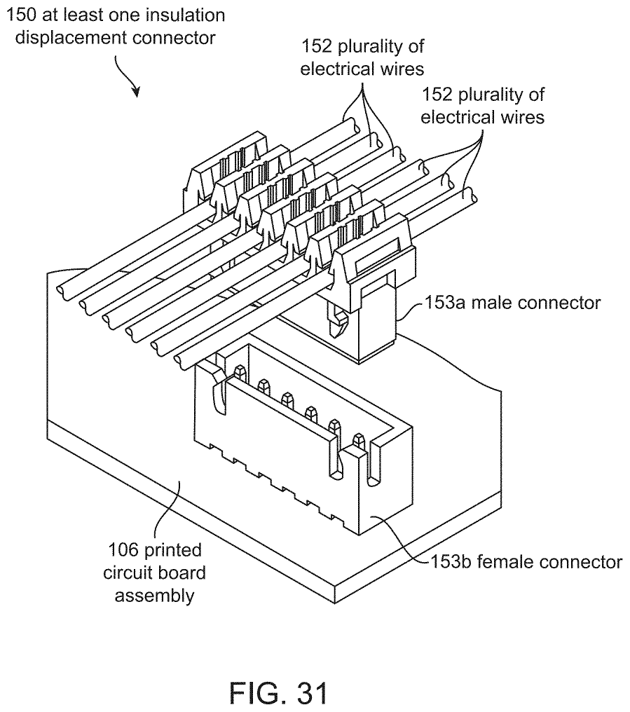

FIG. 31 shows a detailed view of the at least one insulation displacement connector 150. In some embodiments, the at least one insulation displacement connector 150 is configured to receive at least one of power and data from a plurality of electrical wires 152 coupled to the power supply unit 148. The at least one insulation displacement connector 150 may be configured to snapably couple to the PCBA 106 and controller board 132. In some embodiments, the at least one insulation displacement connector 150 comprises a male connector 153a, which is configured to snapably couple to a female connector 153b coupled to the PCBA 106. The male connector 153a may comprise part number 09NR-D4K-P from J.S.T. Mfg. Co., Ltd. The female connector 153b may comprise part number B9B-XH-A from J.S.T. Mfg. Co., Ltd, which may be configured to snapably receive the male connector 153a. In some embodiments, the insulation displacement connector 150 is configured to receive 48V of power from the power supply unit 148 and distribute the power over the plurality of electrical wires 152. The use of the at least one insulation displacement connector 150 may allow the system 100 to make electrical connections and distribute power without losing voltage.

FIG. 32 shows a side view of a portion of the device 100, including the heat sink 158, a thermal interface material, the PCBA 106, the at least one LED 108, the at least one IC driver 146, and a plurality of attachment mechanisms. In some embodiments, the thermal interface material is located between the PCBA 106 and the heat sink 158 to ensure sufficient pressure and interface area between the PCBA 106 and the heat sink 158. As illustrated in FIG. 32, the LEDs 108 and the IC driver 146 may be mounted on the PCBA 106. The light therapy device 102 may include the plurality of attachment mechanisms to mount the PCBA 106 to the heat sink 158. In some embodiments, as shown in FIG. 32, two mounting screws are used are used as the attachment mechanisms. Any number of mounting screws, or other types of attaching mechanisms, may be used to couple the PCBA 106 to the heat sink 158.

FIG. 33 shows a perspective view of the power supply unit 148, including the AC input socket 151. In many embodiments, the power supply unit 148 comprises a medical grade power supply unit 148 at least one of electrically and communicatively coupled to the power button 118. The power supply unit 148 may be configured to output about 48V of power. As previously discussed, the light therapy device 102 may use a single power supply unit 148. The use of a single power supply unit 148 may create a higher efficiency ratio from power consumption to light output. Stated differently, the process of converting power IN to power OUT may be more efficient through the use of a single power supply unit 148 than it would be through the use of multiple power supply units. In addition, the user of a single power supply unit 148 may allow for more light engine options for the system 100. The power supply unit 148 may be double fused for additional safety. The power supply unit 148 may have an operating altitude of up to 5000 meters.

In some embodiments, the power supply unit 148 comprises a medical grade (Class II) power supply unit 148. As such, the device 102 may be considered a Class II medical device. The device 102 may hold the following certifications from the International Electrotechnical Commission: IEC 60601, IEC 60601-1-2, IEC 62471, IEC 60601-2-83, IEC 60601-1-11, IEC 60601-2-57, and IEC 60601-1-6. In some embodiments, the device 102 is a double-insulated device for increased safety.

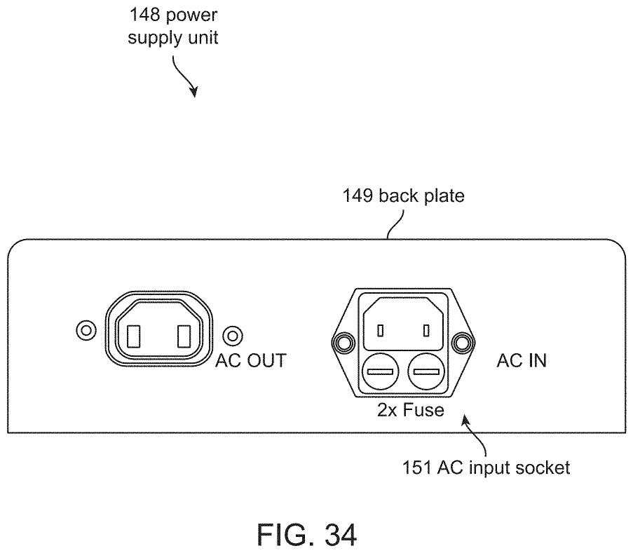

FIG. 34 shows a view of a back plate 149 of the power supply unit 148; including AC input 151 and AC output connections. The AC-IN socket 151 may comprise a C18 type socket with two integrated fuse holders. The AC-OUT socket may comprise a C17 type socket. The power supply unit 148 may include two 250 VAC fuses at 10 A to protect the two lines of the AC-IN socket. The AC input 151 may be configured to plug into a standard 15 A wall socket, then daisy chain to multiple light therapy devices 102 via the RJ11 cables. At least one of the AC-IN 151 and AC-OUT sockets may comprise a locking feature. In some embodiments, all wires, connectors, sockets, and terminals of the power supply unit 148 are UL approved.

### Materials

Any of the components of the light therapy device 10 and the light therapy device 102 may comprise any suitable material or combination of materials. For example, the front bezel 14 of the device 10 may comprise die-cast aluminum, magnesium, or a blend of the two or alloys of the two. The back portion 16 of the device 10 may comprise injection-molded plastic(s). The at least one reflector 30 may comprise clear plastic or glass. The heat sink 158 may comprise aluminum. In many embodiments, the PCBAs 18, 106, comprise a combination of materials.

Various components may comprise any metallic, plastic, or combination material. Metallic materials may comprise steel, aluminum, magnesium, or any other metal or combination of metals. In many embodiments, the components are comprised of durable material(s) configured to withstand everyday wear-and-tear and more significant potential damage, such as being dropped, bumped, knocked against a wall and/or door, etc. The components may comprise any color, whether the natural color of a material (e.g., metallic components may be silver in color) or an applied color (e.g., paint, powder coating, etc.).

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section.

The various features and processes described above may be used independently of one another, or may be combined in various ways. All possible combinations and subcombinations are intended to fall within the scope of this disclosure. In addition, certain methods, events, states, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “substantially” is used to mean “completely” or “nearly completely”. For example, the disclosure includes, “In some embodiments, the front bezel 14 comprises substantially the entire front portion of the device 10.” In this context, “substantially the entire front portion” means that the front bezel may comprise completely or nearly completely the front portion of the device. The front bezel may comprise at least 80% of the front portion of the device, and fall within the understood meaning of “substantially” as used in this disclosure.

The term “about” is used to mean “approximately”. For example, the disclosure includes, “the power supply unit configured to output about 48 volts of power.” In this context, “about 48 volts” means “approximately” 48 volts. A power output between 45 volts and 51 volts may fall within an acceptable range of “about 48 volts”.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A light therapy device, comprising:
a housing comprising a front bezel and a back portion detachably coupled to the front bezel;
a printed circuit board assembly coupled to the housing; and
at least one LED electrically coupled to the printed circuit board assembly, wherein the at least one LED is arranged and configured to emit light through at least one aperture of the front bezel.

2. The light therapy device of claim 1, wherein the front bezel is configured to create a gradient to pull heat from the printed circuit board assembly and thereby act as a heat sink for the light therapy device.

3. The light therapy device of claim 2, wherein the front bezel is comprised of aluminum.

4. The light therapy device of claim 2, wherein an interior surface of the front bezel is coated with a white surface finish to thereby reflect light.

5. The light therapy device of claim 2, further comprising:
at least one lens coupled to the front bezel, the at least one lens positioned to cover at least a portion of the at least one aperture such that the light travels from the at least one LED through the at least one lens; and
at least one reflector coupled to the front bezel, wherein the at least one reflector surrounds at least a portion of the at least one LED to thereby reflect the light emitted from the at least one LED.

6. The light therapy device of claim 1, wherein each LED of the at least one LED comprises a multi-die chip LED comprising four LEDs.

7. The light therapy device of claim 1, wherein the at least one LED is dimmable.

8. The light therapy device of claim 2, wherein the printed circuit board assembly comprises the at least one LED and a control board, and wherein the control board includes at least one integrated circuit driver.

9. The light therapy device of claim 2, further comprising a rechargeable battery that includes a protection circuit module, and wherein the light therapy device is configured to simultaneously charge the rechargeable battery and emit light from the at least one LED.

10. The light therapy device of claim 2, further comprising a rubber handle coupled to a side portion of the housing and configured to enable a user to hold the light therapy device.

11. The light therapy device of claim 2, wherein the housing defines a soap-bar shape ergonomic form factor.

12. The light therapy device of claim 2, wherein the light therapy device is arranged and configured to operate in a recovery plus mode, wherein when the light therapy device operates in the recovery plus mode, the at least one LED is arranged and configured to emit pulsed near infrared light.

13. The light therapy device of claim 2, wherein the light therapy device is arranged and configured to operate in an ambient mode, wherein when the light therapy device operates in the ambient mode, the at least one LED is arranged and configured to emit ambient light.

14. The light therapy device of claim 2, wherein the light therapy device is arranged and configured to operate in an alarm clock mode, wherein when the light therapy device operates in the alarm clock mode, the at least one LED is arranged and configured to emit ambient light at at least one of a predetermined time and upon an occurrence of a predetermined condition, and wherein when the light therapy device operates in the alarm clock mode, the at least one LED is arranged and configured to emit ambient light of increasing intensity.

15. The light therapy device of claim 1, wherein the light therapy device is at least substantially waterproof.

16. A light therapy system including a light therapy device, comprising:
a housing;
a printed circuit board assembly coupled to the housing; and
at least one LED electrically coupled to the printed circuit board assembly, wherein the at least one LED is arranged and configured to emit light through at least one aperture of the housing,
wherein the at least one LED is arranged and configured to pulse at a predetermined frequency and a predetermined duty cycle.

17. The light therapy system of claim 16, further comprising a control panel coupled to the housing and operatively coupled to at least one of the printed circuit board assembly and the at least one LED.

18. The light therapy system of claim 17, wherein the control panel further comprises a power button arranged and configured to operatively control power to the light therapy device.

19. The light therapy system of claim 17, wherein the control panel further comprises a pulse button arranged and configured to operatively control the pulse of the at least one LED.

20. The light therapy system of claim 17, wherein the control panel further comprises at least one of a mode button, a play/pause button, and a time button, wherein the mode button is arranged and configured to select a mode for light emission from the at least one LED, the play/pause button is arranged and configured to at least one of start and stop light emission from the at least one LED, and the time button is arranged and configured to select a time period for light emission from the at least one LED.

21. The light therapy system of claim 17, wherein the control panel further comprises at least one indication light, wherein the at least one indication light is arranged and configured to indicate at least one of a power status, a red light emission status, an infrared light emission status, and a pulse status.

22. The light therapy system of claim 17, wherein the control panel comprises a controller board arranged and configured to operate the control panel, the controller board communicatively coupled to a mobile application on a remote computing device, the mobile application arranged and configured to operate the light therapy device by sending at least one command to the controller board.

23. The light therapy system of claim 17, wherein the control panel further comprises a beeper function arranged and configured to emit a sound after a predetermined amount of time.

24. The light therapy system of claim 16, wherein the printed circuit board assembly comprises the at least one LED and at least one integrated circuit driver.

25. The light therapy system of claim 16, further comprising a medical grade power supply unit at least one of electrically and communicatively coupled to the power button, the power supply unit configured to output about 48 volts of power, wherein the medical grade power supply unit is electrically coupled to at least one insulation displacement connector configured to receive at least one of power and data from a plurality of electrical wires coupled to the medical grade power supply unit.
